SEQUENCE LISTING (2 sequences)

SEQ ID NO: 1 - DNA, 2133 bp, Staphylococcus capitis
atggatttcgtgcctaacaggcacaataagtatgccattagaagatttacagtaggaacggcatcaatattagttggtgcaacattaatattcggagtgaatcatgaagctaaagcggctgagacttcaactgaattaactcaggcacaagcggatgaagattgttcgggtattactgatcaaggccagcaagaagaaatgttaacagaaactcaaaacacacaaaacgactataacgagcaacaaccaactcagcaaatagacaacgattgtattattgatgaagttcctatgaacgaagttgaatatagtgatgatgcatcatccaaagcccaagaagaagatgctacatcattagaaaatgtttcaacagatattaacacacgtaatacggagaatgaatcagttgacgcccaatcaactgataactgcattgcaaatgaacaaacttttgacaacgaatcagtgcaagaacaaacagataaccaagtgaacaacgacaataacatagatgaattacaaaaagcccaagaatatgaaactcaagaagaaaataatgatgcaaatcaatcattgtcagaatcagcagactgtgaaaatgatattcaagcaggttctaacaattacgatatagaagcaataagtggtgtttccgaaaataataatgataatttagataattcatctgatgtctcagctaatggagacgttgctgaaaatgtttcagctttagatagcaactcagattgcgatttatacgcggatcgtagcttagactatgacactgactcaacaagctatgattacaacacagatagtgattacaatacagattgtgactacggctcggatcgtagcttagactatgatactgactcaacaagctatgattacaacacagatagtgattacaatacagattgtgactacggctcggatcgtagcttagactatgatactgattcaacaagttatgattacaacacagatagtggttacgacacagacagtgaatataatactgattgtgattacaacacagatagtgattacaactcagactgtgattatagctcagatagtgattcaggcttagattatgattcagattcaagctacgattcagacgcaagctatgattcagactcaagctacgattcagacgcaagctatgattcagacacagactgtgattacaactcagactgtgattcagactcaagttatgattcagacacagactatgattcagattcagataatgatttagattcagatagcgactcagagtcagattgtgattcggactcagatagcgactcagattcagacagcgattcagactcagatagcgattcagactcagatagcgactcagattcagactgtggttcggattcagactgtgactcagactcagacagcgattcagactcagatagcgactcagactcagatagcgactcagattcagactgtggttcggattcagactgtgactcagactcagatagtgactcagattcagacagcgactcagattcggatagcgactcagattcagacagcgattcagactcagatagtgactcagactcagatagtgactcagattcagatagtgactcagattcagatagcgactcagattcagatagcgactcagattcagactgtggttcggattcagactgtgactcagactcagatagtgactcagactcagatagtgactcagattcagacagcgattcagactcagatagtgattcagactcagactgtggttcggattcagactgtgactcagattcagatagcgattcagactcagacagcgactcagacagcgattcagactcagatagtgactcagattcagacagcggctcaaattgtgattctggttcagaacataaagtaccagtagtaccaacacaatatcatgaaatgacatcacatcatgattcaaaccatcattataataatctagtgatggagcagcatcataagcaagaactaccagatactggttatgatgtggcaaataatggtacgttatttggaggtattcttgcagcattaggatcattacttttagtaggaagcaaacgtagaagtaagaaatactaa

SEQ ID NO: 2 - protein, 710 aa, Staphylococcus capitis
MDFVPNRHNKYAIRRFTVGTASILVGATLIFGVNHEAKAAETSTELTQAQADEDCSGITDQGQQEEMLTETQNTQNDYNEQQPTQQIDNDCIIDEVPMNEVEYSDDASSKAQEEDATSLENVSTDINTRNTENESVDAQSTDNCIANEQTFDNESVQEQTDNQVNNDNNIDELQKAQEYETQEENNDANQSLSESADCENDIQAGSNNYDIEAISGVSENNNDNLDNSSDVSANGDVAENVSALDSNSDCDLYADRSLDYDTDSTSYDYNTDSDYNTDCDYGSDRSLDYDTDSTSYDYNTDSDYNTDCDYGSDRSLDYDTDSTSYDYNTDSGYDTDSEYNTDCDYNTDSDYNSDCDYSSDSDSGLDYDSDSSYDSDASYDSDSSYDSDASYDSDTDCDYNSDCDSDSSYDSDTDYDSDSDNDLDSDSDSESDCDSDSDSDSDSDSDSDSDSDSDSDSDSDSDCGSDSDCDSDSDSDSDSDSDSDSDSDSDSDCGSDSDCDSDSDSDSDSDSDSDSDSDSDSDSDSDSDSDSDSDSDSDSDSDSDSDSDSDSDSDSDSDCGSDSDCDSDSDSDSDSDSDSDSDSDSDSDSDSDSDCGSDSDCDSDSDSDSDSDSDSDSDSDSDSDSDSDSGSNCDSGSEHKVPVVPTQYHEMTSHHDSNHHYNNLVMEQHHKQELPDTGYDVANNGTLFGGILAALGSLLLVGSKRRSKKY

(19) United States
(12) Patent Application Publication
(10) Pub. No.: US 2007/0026011 A1
(43) Pub. Date: Feb. 1, 2007

(54) Sdr proteins from staphylococcus capitis and their use in preventing and treating infections

(75) Inventors: Yule Liu, Rosewell, GA (US); John Vernachio, Canton, GA (US); Joseph Patti, Cumming, GA (US)

Correspondence Address: STITES & HARBISON PLLC, 1199 NORTH FAIRFAX STREET, SUITE 900, ALEXANDRIA, VA, 22314, US

(73) Assignee: INHIBITEX, INC., Alpharetta (GA)

(21) Appl. No.: 10/558,479

(22) PCT Filed: Jun. 1, 2004
(86) PCT No.: PCT/US04/17039

§ 371 Date: Jul. 17, 2006

Related U.S. Application Data
(60) Provisional application No. 60/473,881, filed on May 29, 2003.
(60) Provisional application No. 60/494,550, filed on Aug. 13, 2003.

(51) Int. Cl. A61K 39/085; C07H 21/04; C12N 15/74; C12N 1/21; C07K 14/31

(52) U.S. Cl. 424/190.1; 530/350; 424/243.1; 435/69.3; 435/471; 435/252.3; 536/23.7

(57) ABSTRACT

An isolated and/or purified Sdr surface protein from S. capitis and nucleic acids encoding them are provided which includes the SdrX protein which possesses collagen binding activites and the SdrZL protein which possesses SdrZ-like properties. The Sdr surface proteins from S. capitis can be used in pharmaceutical compositions to treat and prevent S. capitis infectionm and can also be used in vaccines and to raise antibodies which can treat or prevent such infections. Because the SdrX protein has been shown to have collagen binding abilities, antibodies to SdrX will have the ability to inhibit or prevent the ability of S. capitis to bind to collagen.

### CROSS-REFERENCE TO RELATED APPLICATIONS

This application claims the benefit of U.S. Provisional Application Ser. No. 60/494,550, filed Aug. 13, 2003, and Ser. No. 60/473,881 filed May 29, 2003, both applications incorporated herein by reference.

### FIELD OF THE INVENTION

The present invention relates in general to serine-aspartate repeat (Sdr) proteins from Staphylococcus capitis and the nucleic acids coding for them, and in particular to an Sdr protein from S. capitis identified as SdrX along with its A domain which have been discovered to have collagen-binding ability and which thus can be utilized in methods and compositions for treating or preventing Staphylococcal infections.

### BACKGROUND OF THE INVENTION

The successful colonization of the host is a process required for most microorganisms to cause infections in animals and humans. Microbial adhesion is the first crucial step in a series of events that can eventually lead to disease. Pathogenic microorganisms colonize the host by attaching to host tissues or serum conditioned implanted biomaterials, such as catheters, artificial joints, and vascular grafts, through specific adhesins present on the surface of the bacteria. MSCRAMM® proteins are a family of cell-surface adhesins (25) that recognize and specifically bind to distinct extracellular components of host tissues or to serum-conditioned implanted biomaterials such as catheters, artificial joints, and vascular grafts (26). For example, clumping factor (ClfA) is an MSCRAMM® protein expressed by Staphylococcus aureus (S. aureus) that promotes binding of fibrinogen and fibrin to the bacterial cell surface (19, 20). ClfA is the prototype of a multigene family of cell surface proteins characterized by a common domain composed of a unique serine-aspartate repeat region or “Sdr” (18). Other members of this family that are expressed by S. aureus include ClfB (21), SdrC, SdrD, and SdrE (12). Similarly, S. epidermidis expresses a series of Sdr proteins including SdrF, SdrG and SdrH (18). Three additional Sdr family genes have been cloned and sequenced and include SdrY and SdrZ from Staphylococcus caprae, and Sdrl from Staphylococcus saprophyticus, having GenBank accession numbers AY048593, AY048595, and AF402316 respectively.

A number of patents disclose MSCRAMM®s which bind to various extracellular matrix proteins, and these include fibronectin binding proteins such as disclosed in U.S. Pat. Nos. 5,175,096; 5,320,951; 5,416,021; 5,440,014; 5,571,514; 5,652,217; 5,707,702; 5,789,549; 5,840,846; 5,980,908; and 6,086,895; fibrinogen binding proteins such as disclosed in U.S. Pat. Nos. 6,008,341 and 6,177,084; and collagen binding proteins as disclosed in U.S. Pat. Nos. 5,851,794 and 6,288,214; all of these patents incorporated herein by reference.

Because of their critical role in bacterial adhesion, and the expression of these proteins on the bacterial surface, the Sdr family proteins represent attractive targets for immunotherapy. In fact, it has been shown in animal models that ClfA is an excellent target for both active and passive antibody therapies against S. aureus induced sepsis, septic arthritis and endocarditis (31, 13, 7). However, there is a constant and critical need to isolate and identify particular MSCRAMM®s which will be useful in treating or preventing infections caused by key Staphylococcal bacteria most commonly associated with those infections. The Staphylococcus bacteria causes a spectrum of infections that range from cutaneous lesions such as wound infections, impetigo, and furuncles to life-threatening conditions that include pneumonia, septic arthritis, sepsis, endocarditis, and biomaterial related infections.

One particular need for improved treatment is in the field of nosocomial infections. Nosocomial infections result in considerable morbidity and mortality, increased hospitalization, and an increase in healthcare utilization. These infections are especially problematic in premature infants. Late-onset sepsis, an invasive infection occurring in neonates after 72 hours of life, occurs in 21% of very low birth weight (VLBW) infants (29). Coagulase-negative Staphylococcus (CoNS) is considered the leading cause of late-onset infections for this population accounting for 48% of the infections (29). While Staphylococcus epidermidis is often reported as the most frequent isolate among the CoNS causing infections in VLBW infants, still other species of CoNS have been shown to cause sepsis in the susceptible population. A recent report (30) described a bloodstream infection in a premature infant that was caused by S. capitis. In fact, subsequent analysis of blood cultures from neonates between 1997 and 2000 revealed that approximately 20% of the isolates were S. capitis (30). In addition, the S. capitis isolates were heteroresistant to vancomycin. It is thus clear that there is a distinct need to study and identify possible surface proteins from S. capitis which could serve as potential antigenic targets for the development of antibody compositions and therapies, particularly for those compositions and therapies which can treat or prevent nosocomial infections. In addition, because of the uncertainty involved in accurately determining the nature of the surface binding proteins of S. capitis and other adhesins, it is highly desirable to isolate and identify proteins which can be shown to bind to surface proteins such as collagen. Moreover, since antibodies generated against these surface proteins can vary greatly and have a range of effectiveness in inhibiting binding of bacteria to host cells and biological or medical materials and implants, it is important to identify and isolate binding proteins which can generate antibodies that will be effective in blocking such binding and which may be useful in methods of treating or preventing diseases caused by staphylococcal bacteria.

### SUMMARY OF THE INVENTION

Accordingly, it is an object of the present invention to identify and isolate surface proteins from S. capitis which can generate antibodies that will be effective in blocking such binding and which may be useful in methods of treating or preventing diseases caused by staphylococcal bacteria.

It is another object of the present invention to identify and isolate MSCRAMM®s from S. capitis, such as the protein identified as SdrX, as well as their active regions such as the A domain, which can be used to generate monoclonal and polyclonal antibodies that will be useful in methods of treating or preventing infections.

It is further an object of the present invention to provide isolated antibodies that can recognize the A domain of Sdr surface proteins from S. capitis such as the SdrX protein which can be used to inhibit collagen binding and which can be used in compositions and methods of treating or preventing Staphylococcal infections.

It is still further an object of the present invention to utilize the isolated S. capitis Sdr proteins, A domains and antibodies of the invention to produce active and passive vaccines useful in the treatment or prevention of staphylococcal infections, and to provide methods wherein the vaccines and antibodies of the invention are used to prevent or treat a staphylococcal infection.

It is yet another object of the present invention to isolate and identify nucleic acids coding for S. capitis Sdr surface proteins and their A domains and to use these nucleic acids in recombinant methods of producing these surface proteins.

It is yet another object of the invention to isolate plasma donors possessing high antibody titers to the S. capitis Sdr surface proteins and to use such high titer donor plasma in the development of a purified immunoglobulin which can be used to treat or prevent Staphylococcal infections.

These and other objects are provided by virtue of the present invention which comprises identifying, isolating and/or purifying Sdr surface proteins from S. capitis, including the SdrX protein, as well as their immunogenic A domains, and then utilizing these surface proteins in methods of treating and preventing staphylococcal infection. In addition, nucleic acids encoding these proteins and isolated antibodies which recognize these proteins are also provided in accordance with the invention. In particular, the S. capitis Sdr surface protein identified as SdrX has now been determined to be a collagen-binding protein, and antibodies against SdrX have been observed to inhibit the collagen binding activity associated with S. capitis, namely collagen type VI binding activity. In accordance with the invention, pharmaceutical compositions and vaccines can be prepared from Sdr surface proteins from S. capitis which are useful in treating and preventing infections, and the isolated proteins and antibodies recognizing them can be used in methods of diagnosing an infection of S. capitis which employ kits based on those proteins and antibodies. Finally, methods are provided wherein plasma donors may be selected based on a higher than normal antibody titer to Sdr proteins from S. capitis, such as SdrX, and an immunoglobulin product for therapeutic use may be prepared from such selected donor plasma which has a higher than normal antibody titer to an S. capitis Sdr protein.

These embodiments and other alternatives and modifications within the spirit and scope of the disclosed invention will become readily apparent to those skilled in the art from reading the present specification and/or the references cited herein.

### BRIEF DESCRIPTION OF THE DRAWING FIGURES

FIG. 1 shows the identification of a DNA sequence from S. capitis 49326 with homology to the repeat region of sdrG. (A). Schematic representation of the SdrG protein and the region of the probe (arrows). (B). Southern hybridization. The genomic DNAs were digested with HindIII, separated in 1% Agarose gel, and transferred onto Zeta-probe membrane. The blot was hybridized with digoxigenin-labeled probe from the B and the R regions of sdrG. Lane 1, 1 Kb DNA molecular weight marker. Lane 2 and 3, HindlII-digested genomic DNAs from S. epidermidis K28 and S. capitis 49326 respectively. (C). Deduced amino acid sequence of SdrX from S. capitis 49326. The vertical arrow indicates the signal peptide cleavage site. The A region (40aa-254aa) is in bold. The B repeat region (BX) (255aa-420aa) is underlined. The R region (425aa-630aa) containing the SD repetitive sequence is in italics. The cell wall anchoring motif LPDTG (674aa-678aa) is in bold italics.

FIG. 2 is a schematic representation of SdrX in accordance with the invention as compared with previously identified Sdr family members. The relative position and/or size of signal sequences (S), A regions (A), B-repeat regions (BX for SdrX and B for other Sdr members respectively), SD-repeat regions (R), C region (C) (SdrH only), and wall/membrane spanning regions (WM) are shown. The percentage shown on the right indicates the identity of the A region of SdrX to other Sdr family members.

FIG. 3 shows the detection of sdrX mRNA by RT-PCR. Total RNA was isolated from S. capitis 49326 culture at early log, log and stationary phases. 16S rRNA and sdrX RNA were converted into CDNA using sequence specific primers and amplified by RT-PCR. RT− and RT+ indicate without and with reverse transcriptase.

FIG. 4 shows the expression and purification of the A domain of SdrX. The A domain of SdrX was cloned in pQE-30 and expressed as a His-tagged fusion protein in M15[pREP4]. Cell extracts were purified on a chelating HiTrap column. The crude cell extracts before (0 hour) and post (4 hour) induction, the purified protein of 1 μg (P1) and 5 μg (P5) were separated in SDS-PAGE. SeeBluePlus2 was used as molecular weight marker (M).

FIG. 5 shows surface expression of SdrX. A). Detection of surface localization of SdrX by Flow cytometry. The bold line corresponds to early log; the broken line, mid-log; and the dotted line, stationary phase cultures. The grey histogram shows the level of staining with a normal rabbit serum control. B). Western immunoblotting analysis of SdrX. Proteins from cell extracts were separated in 10% Bis-Tris gel in MES/SDS NuPage running buffer, and transferred to PVDF membrane. The western blots were incubated with the hyperimmune serum generated against the A domain of SdrX (r-SdrX) in the presence (right panel) or absence (left panel) of the rSdrX-A as competitor. SeeBluePlus2 was used as molecular weight marker. Lane 1, Cytoplasm fraction from an early log phase culture of S. capitis 35661; Lane 2, 3, and 4. Cell wall fractions from S. capitis 35661 cultures at early log, log, and stationary phases respectively.

FIG. 6 shows the binding of recombinant SdrX (r-SdrX) and whole cell to collagen VI. A. Concentration dependent binding of collagen type VI to immobilized rSdrX-A. Binding to collagen VI (diamond), fibrinogen (triangle), and collagen I (square) to immobilized rSdrX-A. B. Binding of S. capitis strain 35661 to immobilized collagen VI (diamond), fibrinogen (triangle), and BSA (square). C. Inhibition of binding of S. capitis strain 35661 to immobilized collagen VI using rabbit anti-rSdrX A (diamond), or normal rabbit IgG (open square).

FIG. 7 shows SdrX binding to each of the human ECM proteins expressed as absorbance units. Bars correspond to Mean±SD for duplicate measurements.

FIG. 8a shows the nucleotide and amino acid sequences of the SdrZL gene from S. capitis 49326. A putative promoter sequence is shown in bold. The transcription start is in larger font. The ribosome binding (RBS) site is underlined. The arrow indicates the signal peptide cleavage site. The A and C regions are shown. The R region consisting of SD repeat is boxed.

FIG. 8b shows the structural organization of SdrH, SdrZ and SdrZL. Signal sequence (S), A domain (A), SD repeat (SD), and C region (C) and the length are shown. The percentages indicate the similarity to the corresponding regions of SdrZ.

### DETAILED DESCRIPTION OF THE PREFERRED EMBODIMENTS

In accordance with the present invention, the inventors have isolated novel Sdr surface proteins from S. capitis bacteria that can be utilized in methods of treating and preventing bacterial infection, generating an immune response, and in the diagnosis and identification of infections caused by S. capitis. In addition, as described further below, these surface proteins can be used to generate antibodies useful in treating and preventing infection, and also can be utilized in vaccines and pharmaceutical compositions for therapeutic purposes. The present invention further contemplates the use of said Sdr surface proteins from S. capitis in methods of generating immune responses, and treating, diagnosing or preventing an S. capitis infection in the manner as described below. Finally, as described further below, the nucleic acids encoding these surface proteins have also been isolated and sequenced in accordance with the invention.

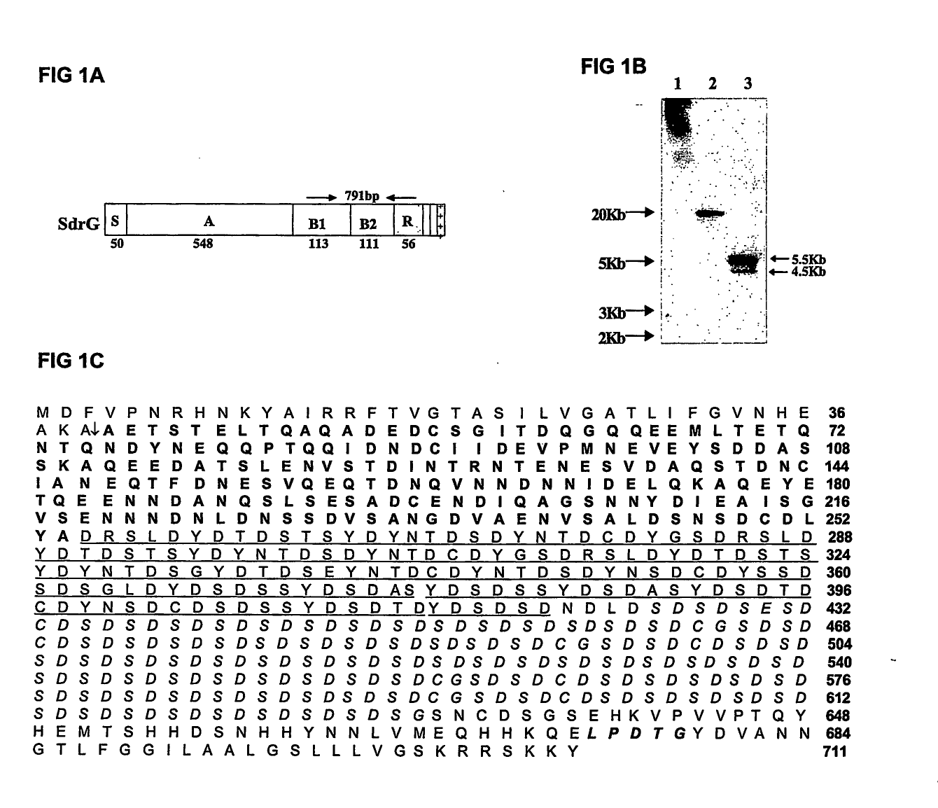

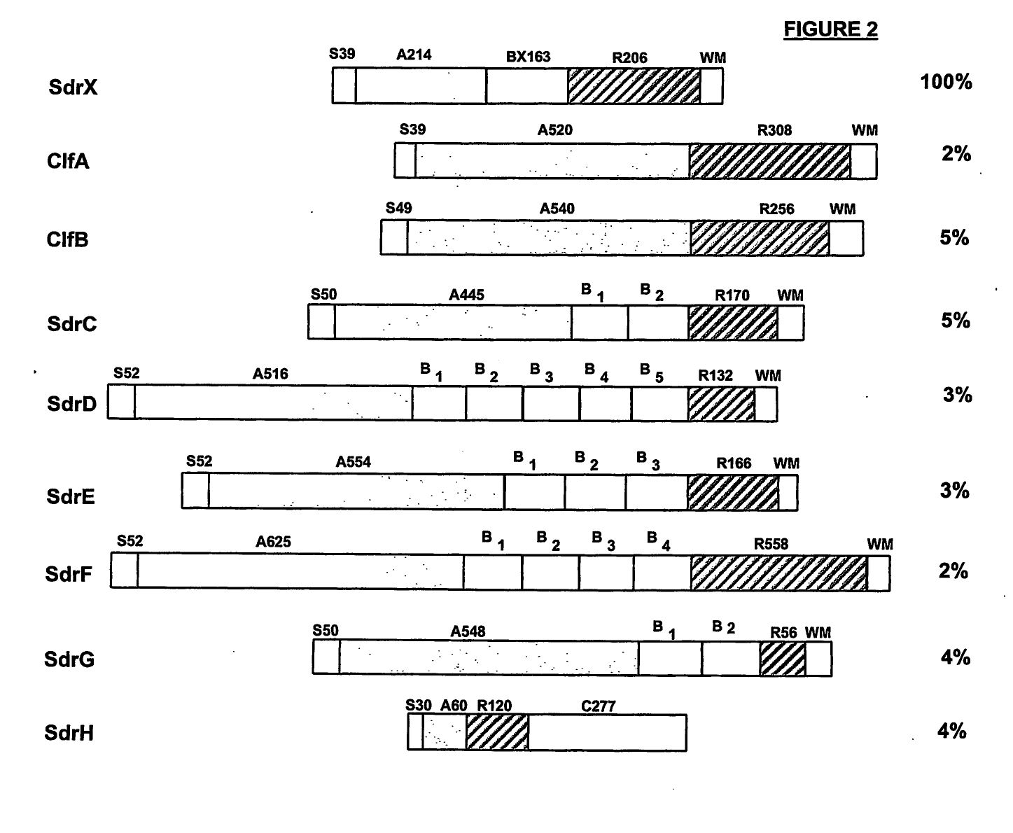

A molecular biology approach was used to determine if Sdr family proteins exist in S. capitis. A DNA fragment corresponding to the B and R regions of the sdrG gene was used to probe the S. capitis genome. As a first surface protein isolated in accordance with the present invention, a novel member of the Sdr family of MSCRAMM®s designated as SdrX was identified, cloned and sequenced. As shown in FIGS. 1 and 2, the deduced protein sequence was compared to the published protein sequences of other Sdr family molecules. The overall structure of the coding region was found to follow the general pattern observed in other Sdr family proteins (18) and included a signal sequence, an A domain, a repetitive domain termed BX, an SD repeat region, a cell wall anchor region with an LPXTG motif sequence (LPDTG amino acids 674-678), a hydrophobic membrane spanning region and a series of positively charged residues at the c-terminus. Individual domains of SdrX were compared to other members of the Sdr family using Clustal W analysis.

Comparison of the SdrX signal sequence showed the greatest homology (˜52%) with SdrC, SdrD, and SdrF. The A domain of SdrX was compared to other Sdr protein sequences and showed little or no homology (less than or equal to 11%). The A domain sequence was also used to perform a BLAST search of the public database at NCBI. Only two protein sequences were found to have homologies greater than 40%, the AtIC protein (44% homology) from S. caprae (1) and the Aas protein (47% homology) a fibronectin-binding autolysin of S. saprophyticus (10). The nature and extent of the relationship of SdrX to either of these proteins is currently not known. Although a conserved sequence (TYTFTDYVD) has been reported to be in the A domains of all S. aureus Sdr proteins (12, 22) and in the S. epidermidis proteins SdrF and SdrG (18), this sequence was not present in the SdrX A-domain. The absence of this sequence suggests that the A-domain of SdrX appears to be of unique structure and unrelated to previously described Sdr protein A-domains.

The repetitive region of 163 amino acids found between the A domain and the SD dipeptide repeat region in SdrX is made up of short repeated sequences varying in length. The repeats are high in S and D content (56% SD overall) but are sufficiently divergent from the dipeptide repeat R region to be categorized as a separate domain. This sequence is considerably divergent from the B regions described in other Sdr proteins. This region in SdrX was therefore named BX to distinguish it from previously described B regions.

The R region of SdrX is typical in size (206 amino acids) for R regions found throughout the Sdr protein family. The presence of this domain places SdrX unequivocally in the Sdr family of staphylococcal proteins.

As shown in FIG. 1C, the amino acid sequence of SdrX is as follows:

| | |
|---|---|
| M D F V P N R H N K Y A I R R F T V | (SEQ ID NO:2) |
| G T A S I L V G A T L I F G V N H E | |
| A K A↓ A E T S T E L T Q A Q A D E | |
| D C S G I T D Q G Q Q E E M L T E T | |
| Q N T Q N D Y N E Q Q P T Q Q I D N | |
| D C I I D E V P M N E V E Y S D D A | |
| S S K A Q E E D A T S L E N V S T D | |
| I N T R N T E N E S V D A Q S T D N | |
| C I A N E Q T F D N E S V Q E Q T D | |
| N Q V N N D N N I D E L Q K A Q E Y | |
| E T Q E E N N D A N Q S L S E S A D | |
| C E N D I Q A G S N N Y D I E A I S | |
| G V S E N N N D N L D N S S D V S A | |
| N G D V A E N V S A L D S N S D C D | |
| L Y A D R S L D Y D T D S T S Y D Y | |
| N T D S D Y N T D C D Y G S D R S L | |
| D Y D T D S T S Y D Y N T D S D Y N | |
| T D C D Y G S D R S L D Y D T D S T | |
| S Y D Y N T D S G Y D T D S E Y N T | |
| D C D Y N T D S D Y N S D C D Y S S | |
| D S D S G L D Y D S D S S Y D S D A | |
| S Y D S D S S Y D S D A S Y D S D T | |
| D C D Y N S D C D S D S S Y D S D T | |
| D Y D S D S D N D L D S D S D S E S | |
| D C D S D S D S D S D S D S D S D S | |
| D S D S D S D S D S D S D C G S D S | |
| D C D S D S D S D S D S D S D S D S | |
| D S D S D S D C G S D S D C D S D S | |
| D S D S D S D S D S D S D S D S D S | |
| D S D S D S D S D S D S D S D S D S | |
| D S D S D S D S D S D S D S D S D S | |
| D C G S D S D C D S D S D S D S D S | |
| D S D S D S D S D S D S D S D S D S | |
| D C G S D S D C D S D S D S D S D S | |
| D S D S D S D S D S D S D S D S D S | |
| G S N C D S G S E H K V P V V P T Q | |
| Y H E M T S H H D S N H H Y N N L V | |
| M E Q H H K Q E [symbol] [symbol] [symbol] [symbol] [symbol] Y D V A N | |
| N G T L F G G I L A A L G S L L L V | |
| G S K R R S K K Y | |

In this deduced sequence, the vertical arrow indicates the signal peptide cleavage site. The A region (40aa-254aa) is in bold. The B repeat region (BX) (255aa-420aa) is underlined. The R region (425aa-630aa) containing the SD repetitive sequence is in italics. The cell wall anchoring motif LPDTG (674aa-678aa) is in bold italics. However, as would be recognized by one of ordinary skill in this art, modification and changes may be made in the structure of the peptides of the present invention and DNA segments which encode them and still obtain a functional molecule that encodes a protein or peptide with desirable characteristics. The amino acid changes may be achieved by changing the codons of the DNA sequence. For example, certain amino acids may be substituted for other amino acids in a protein structure without appreciable loss of interactive binding capacity with structures such as, for example, antigen-binding regions of antibodies or binding sites on substrate molecules. Since it is the interactive capacity and nature of a protein that defines that protein's biological functional activity, certain amino acid sequence substitutions can be made in a protein sequence, and, of course, its underlying DNA coding sequence, and nevertheless obtain a protein with like properties.

It is thus contemplated by the inventors that various changes may be made in the peptide sequences of the disclosed compositions, or corresponding DNA sequences which encode said peptides without appreciable loss of their biological utility or activity. In addition, amino acid substitutions are also possible without affecting the collagen binding ability of the isolated proteins of the invention, provided that the substitutions provide amino acids having sufficiently similar properties to the ones in the original sequences. Accordingly, acceptable amino acid substitutions are generally therefore based on the relative similarity of the amino acid side-chain substituents, for example, their hydrophobicity, hydrophilicity, charge, size, and the like. Exemplary substitutions which take various of the foregoing characteristics into consideration are well known to those of skill in the art and include: arginine and lysine; glutamate and aspartate; serine and threonine; glutamine and asparagine; and valine, leucine and isoleucine. The isolated proteins of the present invention can be prepared in a number of suitable ways known in the art including typical chemical synthesis processes to prepare a sequence of polypeptides.

As shown above, the isolated SdrX amino acid sequence of the present invention contains the conserved sequence motif, LPXTG (18) characteristic of the Sdr family of proteins. This sequence is a substrate for sortase, a transpeptidase that cleaves and covalently links the protein to peptidoglycan in the cell wall, allowing for surface expression of the molecule (17, 28). The sequence LPDTG is found in SdrX at position 674. Taken together, the R region and the BX region provide 419 amino acids between the end of the putative A domain and the LPDTG cell wall anchoring motif. For ClfA, it has been reported that the R region must be 80 residues in length (112 residues in total from A domain to LPXTG) to support wild-type clumping function (9). Therefore, the R region of SdrX would appear to be of sufficient length to allow for exposure of the A domain on the surface of S. capitis. Two lines of evidence demonstrate that SdrX is indeed expressed on the cell surface. By Western blot analysis it was shown that SdrX is present in cell wall fractions from S. capitis but not in cytoplasmic preparations of the same cultures. Also, the A domain of SdrX was found to be accessible to antibodies on the surface of viable S. capitis as measured by flow cytometry. The available data therefore demonstrates that the SdrX protein is a surface expressed protein, as predicted from the primary sequence, and also indicates that the isolated A domain can be used in binding as will be set forth in more detail below. Accordingly, it is contemplated that the SdrX protein in accordance with the invention will encompass SdrX as well as its active fragments such as the SdrX A domain.

Additional tests showed that the C-terminal to the LPDTG sequence is a 23 amino acid sequence containing a high percentage of hydrophobic residues (57%) followed by a short highly polar sequence. Similar regions have been described previously for Sdr proteins (18) and are thought to function as a membrane spanning region and a cytoplasmic tail. Overall, the analysis of the SdrX protein sequence has led to the conclusion that SdrX is a novel member of the Sdr gene family and the first such protein described in S. capitis.

In addition to the sequence data for SdrX, in accordance with the present invention, there is provided nucleic acid sequences which code for SdrX as well as its individual regions including its A domain. The specific nucleic acid sequence encoding the entire SdrX protein was also deduced, and this gene was identified as sdrX. The 2133 nucleotide sequence of sdrX is shown as follows:

| | |
|---|---|
| atggatttcg tgcctaacag gcacaataag | (SEQ ID NO: 1) |
| tatgccatta gaagatttac agtaggaacg | |
| gcatcaatat tagttggtgc aacattaata | |
| ttcggagtga atcatgaagc taaagcggct | |
| gagacttcaa ctgaattaac tcaggcacaa | |
| gcggatgaag attgttcggg tattactgat | |
| caaggccagc aagaagaaat gttaacagaa | |
| actcaaaaca cacaaaacga ctataacgag | |
| caacaaccaa ctcagcaaat agacaacgat | |
| tgtattattg atgaagttcc tatgaacgaa | |
| gttgaatata gtgatgatgc atcatccaaa | |
| gcccaagaag aagatgctac atcattagaa | |
| aatgtttcaa cagatattaa cacacgtaat | |
| acggagaatg aatcagttga cgcccaatca | |
| actgataact gcattgcaaa tgaacaaact | |
| tttgacaacg aatcagtgca agaacaaaca | |
| gataaccaag tgaacaacga caataacata | |
| gatgaattac aaaaagccca agaatatgaa | |
| actcaagaag aaaataatga tgcaaatcaa | |
| tcattgtcag aatcagcaga ctgtgaaaat | |
| gatattcaag caggttctaa caattacgat | |
| atagaagcaa taagtggtgt ttccgaaaat | |
| aataatgata atttagataa ttcatctgat | |
| gtctcagcta atggagacgt tgctgaaaat | |
| gtttcagctt tagatagcaa ctcagattgc | |
| gatttatacg cggatcgtag cttagactat | |
| gacactgact caacaagcta tgattacaac | |
| acagatagtg attacaatac agattgtgac | |
| tacggctcgg atcgtagctt agactatgat | |
| actgactcaa caagctatga ttacaacaca | |
| gatagtgatt acaatacaga ttgtgactac | |
| ggctcggatc gtagcttaga ctatgatact | |
| gattcaacaa gttatgatta caacacagat | |
| agtggttacg acacagacag tgaatataat | |
| actgattgtg attacaacac agatagtgat | |
| tacaactcag actgtgatta tagctcagat | |
| agtgattcag gcttagatta tgattcagat | |
| tcaagctacg attcagacgc aagctatgat | |
| tcagactcaa gctacgattc agacgcaagc | |
| tatgattcag acacagactg tgattacaac | |
| tcagactgtg attcagactc aagttatgat | |
| tcagacacag actatgattc agattcagat | |
| aatgatttag attcagatag cgactcagag | |
| tcagattgtg attcggactc agatagcgac | |
| tcagattcag acagcgattc agactcagat | |
| agcgattcag actcagatag cgactcagat | |
| tcagactgtg gttcggattc agactgtgac | |
| tcagactcag acagcgattc agactcagat | |
| agcgactcag actcagatag cgactcagat | |
| tcagactgtg gttcggattc agactgtgac | |
| tcagactcag atagtgactc agattcagac | |
| agcgactcag attcggatag cgactcagat | |
| tcagacagcg attcagactc agatagtgac | |
| tcagactcag atagtgactc agattcagat | |
| agtgactcag attcagatag cgactcagat | |
| tcagatagcg actcagattc agactgtggt | |
| tcggattcag actgtgactc agactcagat | |
| agtgactcag actcagatag tgactcagat | |
| tcagacagcg attcagactc agatagtgat | |
| tcagactcag actgtggttc ggattcagac | |
| tgtgactcag attcagatag cgattcagac | |
| tcagacagcg actcagacag cgattcagac | |
| tcagatagtg actcagattc agacagcggc | |
| tcaaattgtg attctggttc agaacataaa | |
| gtaccagtag taccaacaca atatcatgaa | |
| atgacatcac atcatgattc aaaccatcat | |
| tataataatc tagtgatgga gcagcatcat | |
| aagcaagaac taccagatac tggttatgat | |
| gtggcaaata atggtacgtt atttggaggt | |
| attcttgcag cattaggatc attactttta | |
| gtaggaagca aacgtagaag taagaaatac | |
| taa | |

This sequence has been deposited in GenBank and has the accession number AY 510088.

Once again, as set forth above, the nucleotide sequences coding for the SdrX protein may have degenerate variations thereof which code for the same sequence of amino acids, as would be recognized by one of ordinary skill in this art. Accordingly, such degenerate nucleic acid sequences are considered part of the present invention.

The identification and isolation of the SdrX protein in accordance with the present invention may proceed via conventional techniques well within the scope of one of ordinary skill in this art, and can use any suitable technique previously known and used to isolate and/or purify other MSCRAMM®s such as disclosed, e.g., in U. S. Pat. Nos. 5,175,096; 5,320,951; 5,416,021; 5,440,014; 5,571,514; 5,652,217; 5,707,702; 5,789,549; 5,840,846; 5,980,908; 6,086,895; 6,008,341; 6,177,084; 5,851,794; 6,288,214; 6,635,473; 6,692,739; and 6,703,025, all of said patents being incorporated herein by reference. In one such suitable procedure, the SdrX gene or its A domain may be cloned using conventional techniques well understood by those of ordinary skill in the art. In one such procedure, cloning of SdrX or its A domain can be conducted using a conventional E. coli process using suitable plasmids such as plasmid pQE-30 and appropriate bacterial strains such as M15[pREP4] (both from Qiagen, Valencia, Cailf.). There are many suitable S. capitis strains available, such as through the ATCC (Manassas, Va.), and in addition, hybridization can be carried out using genomic DNA from an S. epidermidis strain expressing SdrG. Genomic libraries from S. capitis can then be prepared using suitable conventional means and the DNA or products obtained by PCR may then be sequenced. Primers for the gene sdrX can be used in the PCR process, and expression, isolation and/or purification of SdrX or its A domain may occur using any suitable process, such as through a culture of E. coli M15[pREP4] carrying the pQE-30/sdrX or its A domain. A suitable purification process would be one such as the process disclosed in Hall et al., Infect. Immun. 71(12): 6864-6870 (2003), said article incorporated herein by reference. The proteins in accordance with the present invention, including the SdrX protein and it's A domain, can thus be produced recombinantly from nucleic acids encoding them, and it would also be possible to isolate and/or purify natural SdrX and it's A domain from S. capitis if so desired.

Beyond the primary sequence data, further investigation of SdrX and it's A domain have now revealed that unlike any prior Sdr protein, the SdrX protein from S. capitis has been shown to bind to collagen. In particular, screening tests showed that SdrX through its A domain adhered to microtiter plates coated with collagen type VI. Moreover, antibodies generated against the A domain of SdrX are capable of nearly complete abrogation of this binding. Therefore, in accordance with the invention, the SdrX protein is principally responsible for the collagen type VI binding activity of S. capitis and this information can be utilized in order to inhibit the binding of S. capitis to collagen in clinical and therapeutic settings. The fact that antibodies can be generated to inhibit the activity of the SdrX protein evidences that this molecule can be used for the development of antibody therapies against S. capitis infection, as discussed further below.

In accordance with the invention, antibodies are also provided which can recognize the complete SdrX and/or its active fragments such as the A domain, and these antibodies may be monoclonal or polyclonal and can be generated by immunization with an immunogenic portion of SdrX or the SdrX A domain. These antibodies thus may be prepared in any of a number of conventional ways well known to those of ordinary skill in the art. For example, polyclonal antibodies may be produced in conventional ways, such as by introducing an immunogenic amount of SdrX or its A domain into a suitable animal host and then harvesting the antibodies using conventional equipment and techniques. Monoclonal antibodies in accordance with the present invention may be produced, e.g., using the method of Kohler and Milstein (Nature 256:495497, 1975), or other suitable ways known in the field, and in addition can be prepared as chimeric, humanized, or human monoclonal antibodies in ways that would be well known in this field. Still further, monoclonal antibodies may be prepared from a single chain, such as the light or heavy chains, and in addition may be prepared from active fragments of an antibody which retain the binding characteristics (e.g., specificity and/or affinity) of the whole antibody. By active fragments is meant an antibody fragment which has the same binding specificity as a complete antibody which recognizes and binds to the peptide sequences or the proteins of the present invention, and the term “antibody” as used herein is meant to include said fragments. Additionally, antisera prepared using monoclonal or polyclonal antibodies in accordance with the invention are also contemplated and may be prepared in a number of suitable ways as would be recognized by one skilled in the art.

As indicated above, although production of antibodies using recombinant forms of the peptides or proteins of the invention is preferred, antibodies may be generated from natural isolated and purified proteins or peptides as well, and monoclonal or polyclonal antibodies can be generated using the natural peptides or proteins or active regions in the same manner as described above to obtain such antibodies. Still other conventional ways are available to generate the antibodies of the present invention using recombinant or natural purified peptides or proteins or its active regions, as would be recognized by one skilled in the art.

As would be recognized by one skilled in the art, both the proteins and antibodies as described above may be utilized as necessary by forming them into suitable pharmaceutical compositions for administration to a human or animal patient in order to treat or prevent an infection caused by S. capitis. Such pharmaceutical compositions in accordance with the invention may contain, on the one hand, amounts of the SdrX protein or its A domain effective to treat or prevent an S. capitis infection. In addition, the pharmaceuticals may also be prepared which contain effective amounts the antibodies of the present invention, or effective fragments thereof. In either case, these compositions are formulated in combination with any suitable pharmaceutical vehicle, excipient or carrier that would commonly be used in this art, including such as saline, dextrose, water, glycerol, ethanol, other therapeutic compounds, and combinations thereof. As one skilled in this art would recognize, the particular vehicle, excipient or carrier used will vary depending on the patient and the patient's condition, and a variety of modes of administration would be suitable for the compositions of the invention, as would be recognized by one of ordinary skill in this art. Suitable methods of administration of any pharmaceutical composition disclosed in this application include, but are not limited to, topical, oral, anal, vaginal, intravenous, intraperitoneal, intramuscular, subcutaneous, intranasal and intradermal administration.

For topical administration, compositions may be formulated in the form of an ointment, cream, gel, lotion, drops (such as eye drops and ear drops), or solution (such as mouthwash). Wound or surgical dressings, sutures and aerosols may be impregnated with the composition. The composition may contain conventional additives, such as preservatives, solvents to promote penetration, and emollients. Topical formulations may also contain conventional carriers such as cream or ointment bases, ethanol, or oleyl alcohol.

When necessary, the pharmaceutical compositions of the present invention may also be administered with a suitable adjuvant in an amount effective to enhance the immunogenic response against the conjugate. For example, suitable adjuvants may include alum (aluminum phosphate or aluminum hydroxide), which is used widely in humans, and other adjuvants such as saponin and its purified component Quil A, Freund's complete adjuvant, RIBBI adjuvant, and other adjuvants used in research and veterinary applications. Still other chemically defined preparations such as muramyl dipeptide, monophosphoryl lipid A, phospholipid conjugates such as those described by Goodman-Snitkoff et al. J. Immunol. 147:410-415 (1991) and incorporated by reference herein, encapsulation of the conjugate within a proteoliposome as described by Miller et al., J. Exp. Med. 176:1739-1744 (1992) and incorporated by reference herein, and encapsulation of the protein in lipid vesicles such as Novasomem™ lipid vesicles (Micro Vescular Systems, Inc., Nashua, N.H.) may also be useful.

In any event, the pharmaceutical compositions of the present invention will thus be useful for treating or preventing infections caused by S. capitis and also in reducing or eliminating the binding of these bacteria to collagen.

As indicated above, in accordance with the present invention, methods are provided for preventing or treating an S. capitis bacterial infection which comprise administering an SdrX protein such as SdrX or its A domain, or an antibody in accordance with the invention as set forth above in amounts effective to treat or prevent the infection. Accordingly, in accordance with the invention, administration of the proteins, antibodies or pharmaceutical compositions of the present invention may occur in any of the conventional ways described above (e.g., topical, parenteral, intramuscular, etc.), and will thus provide an extremely useful method of treating or preventing S. capitis bacterial infections in human or animal patients. By effective amount is meant that level of use, such as of an antibody titer, that will be sufficient to prevent, treat or reduce an S. capitis infection, or that amount by which adherence or binding of the S. capitis bacteria to collagen will be inhibited which will also be useful in the treatment or prevention of S. capitis bacterial infections. As would be recognized by one of ordinary skill in this art, the level of antibody titer needed to be effective in treating or preventing a particular S. capitis infection will vary depending on the nature and condition of the patient, and/or the severity of the pre-existing infection.

In addition to the treatment or prevention of S. capitis bacterial infection, the present invention contemplates the use of the proteins and antibodies of the invention in the detection and diagnosis of such an infection, whether in a patient or on medical equipment which may also become infected. In accordance with the invention, a preferred method of detecting the presence of such infections involves the steps of obtaining a sample suspected of being infected by one or more S. capitis bacteria species or strains, such as a sample taken from an individual, for example, from one's blood, saliva, tissues, bone, muscle, cartilage, or skin. In one method, SdrX or its A domain can be used to detect antibodies to S. capitis using a conventional kit or assay. In this form of the invention, a suitable kit may include SdrX or its A domain along with a means to introduce a sample suspected of containing S. capitis antibodies and a means for detecting binding of the antibodies in the sample to the SdrX antigens following sufficient time for binding to take place.

Alternatively, the kit may be prepared using the isolated SdrX antibodies as disclosed above, and this diagnostic kit will generally contain the SdrX antibody, means for introducing the antibody to a sample suspected of containing S. capitis bacteria or bacterial proteins, and means for detecting binding of the sample to the antibodies following a sufficient time for binding to take place. Accordingly, in accordance with the invention, a method of diagnosing a S. capitis bacterial infection is contemplated wherein a sample suspected of being infected with such bacteria has added to it an antibody in accordance with the present invention, and a S. capitis bacterial infection will be indicated by antibody binding to the appropriate proteins or peptides in the sample.

In certain cases, the antibody or antigen in the kits will be conjugated to a detectable label for purposes of determining the presence of the respective binding partner to said antibody or antigen in the sample. For example, the antibody or antigen in the kit can be conjugated (directly or via chelation) to a radiolabel such as, but not restricted to, ³²P, ³H, ¹⁴C, ³⁵S, ¹²⁵I, or ¹³¹I. Detection of a label can be by methods such as scintillation counting, gamma ray spectrometry or autoradiography. Bioluminescent labels, such as derivatives of firefly luciferin, are also useful. The bioluminescent substance is covalently bound to the protein by conventional methods, and the labeled protein is detected when an enzyme, such as luciferase, catalyzes a reaction with ATP causing the bioluminescent molecule to emit photons of light. Fluorogens may also be used to label proteins. Examples of fluorogens include fluorescein and derivatives, phycoerythrin, allo-phycocyanin, phycocyanin, rhodamine, and Texas Red. The fluorogens are generally detected by a fluorescence detector.

Accordingly, antibodies in accordance with the invention may be used for the specific detection of S. capitis bacterial or surface proteins, for the prevention of infection from S. capitis bacteria, for the treatment of an ongoing infection, or for use as research tools. The term “antibodies” as used herein includes monoclonal, polyclonal, chimeric, single chain, bispecific, simianized, and humanized or primatized antibodies as well as Fab fragments, such as those fragments which maintain the binding specificity of the antibodies to the peptides and/or proteins of the present invention, including the products of an Fab immunoglobulin expression library. Accordingly, the invention contemplates the use of single chains such as the variable heavy and light chains of the antibodies as set forth above. Generation of any of these types of antibodies or antibody fragments is well known to those skilled in the art.

As indicated above, antibodies or antigens used in the kits in accordance with the invention may be labeled directly with a detectable label for identification and quantification of S. capitis bacteria. Labels for use in immunoassays are generally known to those skilled in the art and include enzymes, radioisotopes, and fluorescent, luminescent and chromogenic substances, including colored particles such as colloidal gold or latex beads. Suitable immunoassays include enzyme-linked immunosorbent assays (ELISA).

Alternatively, the label may be provided indirectly by reaction with labeled substances that have an affinity for immunoglobulin. The antibody or antigen may be conjugated with a second substance and detected with a labeled third substance having an affinity for the second substance conjugated to the antibody. For example, the antibody or antigen may be conjugated to biotin and the antibody-biotin conjugate detected using labeled avidin or streptavidin. Similarly, the antibody or antigen may be conjugated to a hapten and the antibody-hapten conjugate detected using labeled anti-hapten antibody. These and other methods of labeling antibodies or antigens and assay conjugates are well known to those skilled in the art.

In addition, as set forth above, there may be cases, such as where the patient is a human, wherein it may be preferred that the antibody is “humanized” by transplanting the complimentarity determining regions of the hybridomaderived antibody into a human monoclonal antibody as described, e.g., by Jones et al, Nature 321:522-525 (1986) or Tempest et al. Biotechnology 9:266-273 (1991) or “veneered” by changing the surface exposed murine framework residues in the immunoglobulin variable regions to mimic a homologous human framework counterpart as described, e.g., by Padlan, Molecular Imm. 28:489-498 (1991), or European Patent application 519,596, these references incorporated herein by reference. Even further, when so desired, the monoclonal antibodies of the present invention may be administered in conjunction with a suitable antibiotic to further enhance the ability of the present compositions to fight bacterial infections.

In another embodiment of the invention, there is provided active or passive vaccines based on SdrX or its A domain or antibodies thereto. In accordance with the invention, an active vaccine may be constructed which comprises an immunogenic or effective amount of the complete SdrX protein or the SdrX A domain combined with a pharmaceutically acceptable vehicle, carrier or excipient. By immunogenic amount is considered to be that amount which will give rise to an immunological reaction in the patient whereby antibodies to SdrX or its A domain are produced, and this amount will differ depending on the nature and condition of the patient as well as the mode of administration. A passive vaccine is also provided which comprises antibodies as described above in combination with a pharmaceutically acceptable vehicle, carrier or excipient, and the passive vaccine will include an effective amount of the antibodies so as to be useful to treat or prevent an S. capitis bacterial infection.

As would be recognized by one skilled in this art, such a vaccine may be packaged for administration in a number of suitable ways, such as by parenteral (i.e., intramuscular, intradermal or subcutaneous) administration or nasopharyngeal (i.e., intranasal) administration. One such mode is where the vaccine is injected intramuscularly, e.g., into the deltoid muscle. However, the particular mode of administration will depend on the nature of the bacterial infection to be dealt with and the condition of the patient. The vaccine is preferably combined with a pharmaceutically acceptable vehicle, carrier or excipient to facilitate administration, and such a vehicle, carrier or excipient may be water or a buffered saline, with or without a preservative. The vaccine may be lyophilized for resuspension at the time of administration or in solution.

The preferred dose for administration of an antibody composition in the passive vaccines in accordance with the present invention is that amount which will be effective in preventing or treating an S. capitis bacterial infection, and one would readily recognize that this amount will vary greatly depending on the nature of the infection and the condition of a patient. As indicated above, an “effective amount” of antibody or pharmaceutical agent to be used in accordance with the invention is intended to mean a nontoxic but sufficient amount of the agent, such that the desired prophylactic or therapeutic effect is produced. Thus, the exact amount of the antibody or a particular agent that is required will vary from subject to subject, depending on the species, age, and general condition of the subject, the severity of the condition being treated, the particular carrier or adjuvant being used and its mode of administration, and the like. Accordingly, the “effective amount” of any particular antibody composition will vary based on the particular circumstances, and an appropriate effective amount may be determined in each case of application by one of ordinary skill in the art using only routine experimentation. The dose should be adjusted to suit the individual to whom the composition is administered and will vary with age, weight and metabolism of the individual. The compositions may additionally contain stabilizers or pharmaceutically acceptable preservatives, such as thimerosal (ethyl(2-mercaptobenzoate-S)mercury sodium salt) (Sigma Chemical Company, St. Louis, Mo.).

The immunological compositions, such as vaccines, and other pharmaceutical compositions can be used alone or in combination with other blocking agents to protect against human and animal infections caused by or exacerbated by staphylococci. For example, the compositions may be effective against a variety of conditions, including use to protect humans against skin infections such as impetigo and eczema, as well as mucous membrane infections such as tonsillopharyngitis. In addition, effective amounts of the compositions of the present invention may be used to protect against complications caused by localized infections such as sinusitis, mastoiditis, parapharygeal abscesses, cellulitis, necrotizing fascitis, myositis, streptococcal toxic shock syndrome, pneumonitis endocarditis, meningitis, osteomylitis, and many other sever diseases. Further, the present compositions can be used to protect against nonsuppurative conditions such as acute rheumatic fever, acute glomerulonephritis, and exacerbations of forms of psoriasis such as psoriasis vulgaris. The compositions may also be useful as appropriate in protecting both humans and other species of animals where needed to combat similar staphylococcal infections.

To enhance immunogenicity, the proteins may be conjugated to a carrier molecule. Suitable immunogenic carriers include proteins, polypeptides or peptides such as albumin, hemocyanin, thyroglobulin and derivatives thereof, particularly bovine serum albumin (BSA) and keyhole limpet hemocyanin (KLH), polysaccharides, carbohydrates, polymers, and solid phases. Other protein derived or non-protein derived substances are known to those skilled in the art. An immunogenic carrier typically has a molecular weight of at least 1,000 Daltons, preferably greater than 10,000 Daltons. Carrier molecules often contain a reactive group to facilitate covalent conjugation to the hapten. The carboxylic acid group or amine group of amino acids or the sugar groups of glycoproteins are often used in this manner. Carriers lacking such groups can often be reacted with an appropriate chemical to produce them. Preferably, an immune response is produced when the immunogen is injected into animals such as mice, rabbits, rats, goats, sheep, guinea pigs, chickens, and other animals, most preferably mice and rabbits. Alternatively, a multiple antigenic peptide comprising multiple copies of the protein or polypeptide, or an antigenically or immunologically equivalent polypeptide may be sufficiently antigenic to improve immunogenicity without the use of a carrier.

The SdrX protein, or active portions thereof, or combination of proteins, may be administered with an adjuvant in an amount effective to enhance the immunogenic response against the conjugate. For example, an adjuvant widely used in humans has been alum (aluminum phosphate or aluminum hydroxide). Saponin and its purified component Quil A, Freund's complete adjuvant and other adjuvants used in research and veterinary applications have toxicities which limit their potential use in human vaccines. However, chemically defined preparations such as muramyl dipeptide, monophosphoryl lipid A, phospholipid conjugates such as those described by Goodman-Snitkoff et al. J Immunol. 147:410-415 (1991) and incorporated by reference herein, encapsulation of the conjugate within a proteoliposome as described by Miller et al., J. Exp. Med. 176:1739-1744 (1992) and incorporated by reference herein, and encapsulation of the protein in lipid vesicles such as Novasome™ lipid vesicles (Micro Vescular Systems, Inc., Nashua, N.H.) may also be useful.

The term “vaccine” as used herein includes not only vaccines comprising SdrX proteins but of nucleic acids coding for the SdrX which may also be used in a pharmaceutical composition that may be administered to a patient. For genetic immunization, suitable delivery methods known to those skilled in the art include direct injection of plasmid DNA into muscles (Woff et al., Hum. Mol. Genet. 1:363, 1992), delivery of DNA complexed with specific protein carriers (Wu et al., J Biol. Chem. 264:16985, 1989), coprecipitation of DNA with calcium phosphate (Benvenisty and Reshef, Proc. Natl. Acad. Sci. 83:9551, 1986), encapsulation of DNA in liposomes (Kaneda et al., Science 243:375, 1989), particle bombardment (Tang et al., Nature 356:152, 1992 and Eisenbraun et al., DNA Cell Biol. 12:791, 1993), and in vivo infection using cloned retroviral vectors (Seeger et al., Proc. Natl. Acad. Sci. 81:5849, 1984).

There are several advantages of immunization with a gene rather than its gene product. The first is the relative simplicity with which native or nearly native antigen can be presented to the immune system. Mammalian proteins expressed recombinantly in bacteria, yeast, or even mammalian cells often require extensive treatment to ensure appropriate antigenicity. A second advantage of DNA immunization is the potential for the immunogen to enter the MHC class I pathway and evoke a cytotoxic T cell response. Immunization of mice with DNA encoding the influenza A nucleoprotein (NP) elicited a CD8⁺ response to NP that protected mice against challenge with heterologous strains of flu. (See Montgomery, D. L. et al., Cell Mol Biol, 43(3):285-92, 1997 and Ulmer, J. et al., Vaccine, 15(8):792-794, 1997.)

As indicated above, both the proteins and the antibodies of the present invention, or active portions or fragments thereof, are particularly useful for fighting or preventing bacteria infection in patients or on in-dwelling medical devices to make them safer for use. Such medical devices may include vascular grafts, vascular stents, intravenous catheters, artificial heart valves, cardiac assist devices and other medical devices or implants which may themselves be susceptible to bacterial infestation. In short, the proteins and antibodies of the present invention are thus extremely useful in treating or preventing S. capitis infections in human and animal patients and in medical or other in-dwelling devices.

The SdrX protein, or active fragments thereof, are useful in a method for screening compounds to identify compounds that inhibit collagen binding of staphylococci to host molecules. In accordance with the method, the compound of interest is combined with one or more of the SdrX proteins or fragments thereof and the degree of binding of the protein to collagen or other extracellular matrix proteins is measured or observed. If the presence of the compound results in the inhibition of protein-collagen binding, for example, then the compound may be useful for inhibiting staphylococci in vivo or in vitro. The method could similarly be used to identify compounds that promote interactions of staphylococci with host molecules. The method is particularly useful for identifying compounds having bacteriostatic or bacteriocidal properties.

In accordance with the present invention, the SdrX proteins as described above, including SdrX, or the SdrX A domain, or active fragments from SdrX, may also be utilized in the development of vaccines for immunization against S. capitis infections, and thus a method of eliciting an immune response in a human or animal is also provided wherein an immunogenic amount of an SdrX protein in accordance with the invention is administered to a human or animal. In the preferred embodiment, vaccines in accordance with the invention are prepared using methods that are conventionally used to prepare vaccines, and the preferred vaccine comprises an immunogenic amount of the peptides or proteins as described above along with a pharmaceutically acceptable vehicle, carrier or excipient.

The present invention thus provides for the identification and isolation of proteins having the signature conserved regions as set forth above, as well as the vaccines, antibodies and other forms of the invention as set forth above, and the invention will be particularly useful in developing and administering treatment regimens which can be used to fight or prevent infections caused by S. capitis bacteria. In general, the invention thus also comprises a method of treating or preventing an S. capitis infection in a human or animal patient in need of such treatment comprising administering to the patient the isolated SdrX protein or antibody thereto in an amount effective to treat or prevent such an infection.

In addition to the above methods, a method of obtaining a purified donor immunoglobulin containing a higher than normal antibody titer to an SdrX protein which comprises obtaining donor plasma from individuals, screening the donor plasma to identify those donors having higher than normal antibody titers to the SdrX protein, and collecting donor plasma from said high-titer individuals and purifying the immunoglobulin so as to provide an immunoglobulin product having a higher than normal antibody titer to the SdrX protein than that which would be obtained by normal pooled donor plasma. Alternatively, a purified immunoglobulin having a higher than normal antibody titer to the SdrX protein may be obtained by first stimulating selected donors with an immunogenic amount of the SdrX protein in accordance with the invention so that the donor develops a higher than normal antibody titer to SdrX, and then obtaining the purified immunoglobulin from said stimulated donors. These methods and purified immunoglobulin products include the types of methods and products disclosed with regard to other staphylococcal adhesins in U.S. Pat. No. 6,692,739, incorporated herein by reference.

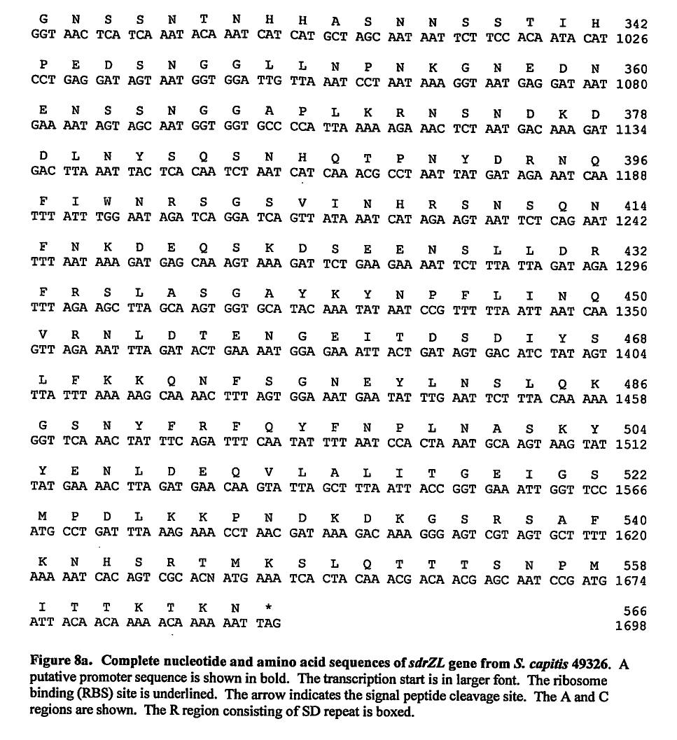

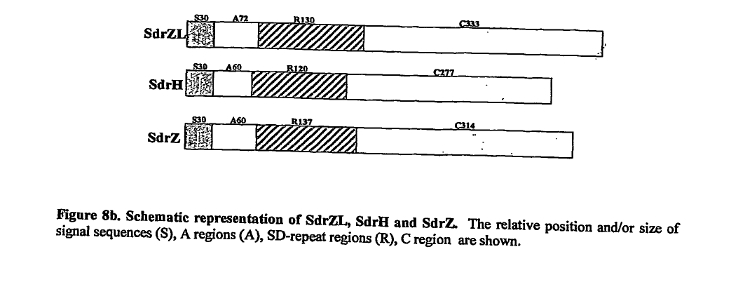

In another aspect of the present invention, a second surface Sdr protein from S. capitis has been obtained using the isolation techniques described above, and this protein has been identified as SdrZL since it is an “SdrZ-like” protein. The details and sequences of this protein and its nucleic acid (sdrZL) are provided herein and are best shown in FIGS. 8a and 8b. In accordance with the present invention, it is contemplated that SdrZL may also be used to treat, diagnose or prevent an infection from S. capitis in a similar manner to the SdrX protein as described above, and thus can be utilized as a pharmaceutical composition or vaccine as set forth above with regard to SdrX, can be used to generate antibodies thereto which can recognize the SdrZL protein, and can be used in all of the above methods and kits as described above.

While the invention has been described above with regard to preferred embodiments, it is clear to one skilled in the art that there will be additional embodiments, compositions and methods which fall within the scope of the invention which have not been specifically described above.

The following examples are provided which exemplify aspects of the preferred embodiments of the present invention. However, it will be appreciated by those of skill in the art that the techniques disclosed in the example which follow represent techniques discovered by the inventors to function well in the practice of the invention, and thus can be considered to constitute preferred modes for its practice. Moreover, those of skill in the art will also appreciate that in light of the present specification, many changes can be made in the specific embodiments which are disclosed and still obtain a like or similar result without departing from the spirit and scope of the invention.

### EXAMPLES

### Example 1

Identifying and Isolating the SdrX Protein

Overview

To determine if additional members of the Sdr protein family are present in S. capitis a gene fragment incorporating the Sdr repeat region of the S. epidermidis sdrG gene was used to probe the S. capitis genome. In the present study, we report the identification and characterization of a novel Sdr family protein from S. capitis. The data demonstrate that this new gene, sdrX, encodes a surface expressed protein with sequence motifs in common with other Sdr proteins from staphylococci. Additionally, SdrX was found to be the first Sdr protein to bind collagen and antibodies against SdrX were shown to inhibit collagen type VI binding activity associated with S. capitis.

Materials and methods

Bacterial strains and plasmids. Escherichia coli strain XL10-Gold ultra-competent cells (Stratagene, LaJolla Cailf.) and Topo10F′ competent cells (Invitrogen, Carlsbad, Calif.) were used as hosts for DNA transformation. Plasmid pUC18 was used for cloning of genomic DNA fragments. Plasmid pQE-30 (Qiagen, Valencia, Calif.) was used for cloning the A domain of SdrX. The bacterial strain M15[pREP4] (Qiagen, Valencia, Calif.) was used for expression of the recombinant SdrX A domain. S. capitis strains 27840, 27841, 27842, 27843, 35661, 49324, 49325, 49326 and 49327 were obtained from the American Type Culture Collection (ATCC, Manassas, Va.). S. capitis strains 004102 and 012106 were clinical isolates from NICU patients. S. epidermidis strain K28 was a gift from Dr. M. Hook.

Southern hybridization. Genomic DNA from S. epidermidis K28 and S. capitis 49326 was prepared using the G/Nome DNA kit, (Bio 101, Carlsbad, Cailf.) with the addition of 2 mg/ml lysozyme and 0.1 mg/ml lysostaphin (Sigma, St. Louis, Mo.) to the cell suspension solution. The hybridization probe was made from the genomic DNA of S. epidermidis K28 by PCR and labeled with digoxigenin (Roche Applied Science, Indianapolis, Ind.). The PCR primers span the B and R regions of sdrG (forward primer, 5′-CCGCTTAGTAATGTATTG-3′; reverse primer, 5′-TCTTATCTGAGCTATTG-3′). For Southern blotting, 1 μg of genomic DNA was digested with 20 U of HindIII at 37° C. overnight and separated in a 0.8% agarose gel. The southern transfer, hybridization, and washing were performed according to the instruction manual for Zeta-probe GT Blotting Membrane (Bio-Rad, Hercules, Cailf.) except that the hybridization and washing were done at 45° C. After washing, the membrane was incubated with the anti-digoxigenin-POD antibody (Roche Applied Science, Indianapolis, Ind.) and the signal was detected with Supersignal West Pico Chemiluminescent Substrate (Pierce, Rockford, Ill).

Genomic DNA Library Preparation and Screening. Genomic DNA from S. capitis 49326 was digested with HindIII and separated in a 0.8% agarose gel. DNA fragments ranging from 4 to 6 Kb were purified from the gel, ligated into HindIII digested pUC18, and transformed into XL10 Gold ultra-competent E. coli (Stratagene, LaJolla, Calif.). The bacterial colonies were blotted onto 85 mm C/P Lift Membrane (BioRad, Hercules, Cailf.) and lysed with 0.5 N NaOH,1% SDS for 10 min. The membrane was then washed with 2×SSC and baked at 80° C. for 30 min. Colony hybridization was performed with the digoxigenin-labeled hybridization probe under the same conditions as for the Southern hybridization.

DNA sequencing and analysis. The cloned DNA fragments or PCR products were sequenced by primer extension sequencing (Seqwright, Houston, Tex.). DNA and amino acid sequences were analyzed using Lasergene software (DNASTAR, Inc., Madison, Wis.). The BLAST network service (http://www.ncbi.nlm.nih.gov/) was used for sequence homology searches.

Genomic DNA PCR. Genomic DNA was prepared from log phase cultures using the MicroLysis kit (Microzone Ltd., West Sussex, UK). Lysis of bacterial cells was achieved through 3 thermal cycles (65° C., 5 min, 96° C., 2 min, 65° C., 4 min, 96° C., 1 min, 65° C., 1 min, 96° C., 30 Seconds, in GeneAmp PCR system 2400 (Perkin Elmer, Wellesley, Mass.). The clarified supematant was collected and amplified by PCR for 30 cycles at 94° C., 30 seconds, 47° C., 30 seconds, 72° C., 1 minute using primers specific for sdrX (sdrX-AF, 5′-CGGGATCCGAGACTTCAACTGAATTAAC-3′ and sdrX-AR, 5′-AACTGCAGCGCGTATAAATCGCAATCTG-3′).

Reverse transcription (RT)-PCR analysis of sdrX expression. Total RNA was isolated from early log, log and stationary phases of bacteria cultures as follows. Two volumes of the RNAprotect Bacteria Reagent (Qiagen, Valencia, Calif.) were added to one volume of cell culture, and treated at room temperature for 5 min. Total RNA was isolated using the RNeasy Mini Kit (Qiagen, Valencia, Calif.) according to the manufacturer's instructions, except that cells were lysed with 2 mg/ml lysozyme and 0.1 mg/ml lysostaphin (Sigma, St. Louis, Mo.) for 1 hr at 37° C. Reverse transcription was carried out with MLV reverse transcriptase (Promega, Madison, Wis.) in the presence of dNTPs, 2 μg of total RNA, and the primer for sdrX (5′-AACTGCAGCGCGTATAAATCGCAATCTG-3′) or 16SrRNA (5′-AACTTTATGGGATTTGCT-3′). The resulting cDNA was amplified by PCR with primers specific for 16S RNA and sdrX. (16S rRNA primers: 5′-TTGAAACTCAAAGGAATTG-3′ and 5′-AACTTTATGGGATTTGCT-3′) (sdrX primers: 5′-GGTATGCCATTAGMGATTTAC-3′ and 5′-AACTGCAGCGCGTATAAATCGCAATCTG-3′).

Cloning the SdrX A domain. The A domain of SdrX was amplified by PCR from the genomic DNA of S. capitis 49326 (forward primer: 5′-CGGGATCCGAGACTTAACTGAATTAAC-3′; reverse primer: 5′-AACTGCAGCGCGTATAAATCGCAATCTG-3′). PCR was carried out with pfu Turbo DNA polymerase (Stratagene, La Jolla, Calif.) for 30 cycles at 94° C., 30 seconds, 45° C., 30 seconds, 72° C., 1 min. The PCR product was gel purified using the Qiaquick Gel Extraction kit (Qiagen, Valencia, Calif.), digested with BamHI and PstI, and ligated into pQE-30 (Qiagen, Valencia, Calif.) using T4 DNA ligase (New England Biolabs, Beverly, Mass.). The resulting plasmid (pQE-30/sdrX-A) was transformed into bacterial strain M15[pREP4], and the transformants were selected on Luria broth plates supplemented with ampicillin (100 μg/ml) and kanamycin (25 μg/ml) (Sigma, St. Louis, Mo.).

Expression and purification of the SdrX A domain. E. coli M15[pREP4] carrying pQE-30/sdrX-A was cultured in Luria broth supplemented with ampicillin (100 μg/ml) and kanamycin (25 μg/ml) at 37° C. to OD₆₀₀ₙₘ of 0.9. Gene expression was induced with 1 mM IPTG for 3 hours. Cells were harvested and resuspended in the lysis buffer containing 25 mM Tris (pH 8.0), 0.5M NaCl and 5 mM imidazole. The recombinant protein was purified as previously described (7).

Preparation of polyclonal antiserum and purified hyperimmune antibodies. The purified recombinant SdrX A domain protein was used as an immunogen to generate polyclonal antiserum in both mice and rabbits. Serum was separated from blood collections and for some applications the IgG fraction from the serum was purified using Protein G affinity chromatography.

Flow Cytometry. The expression of SdrX by S. capitis cells in early log phase, log phase and overnight cultures was determined by flow cytometry using a FACSCalibur Flow Cytometer (B-D Biosciences, San Jose, Calif.) equipped with an argon-ion laser (488 nm) as previously described (7).

Detection of SdrX by Western blot analysis. S. capitis cells from early log phase, log phase and overnight cultures were washed in water and resuspended in the cell suspension solution (G/Nome DNA kit, Bio-101, Carlsbad, Calif.) containing 1× proteinase inhibitor cocktail (PIC) (Sigma, St. Louis, Mo.). Bacteria were lysed by sonication for 5 times, 10 seconds each using the Sonic Dismembrator 550 (Fisher Scientific, Hampton, N.H.). The lysate was cleared by centrifugation at 20800×g for 10 min. The supematant was collected as the cytoplasm fraction. The pellet was resuspended in the cell suspension solution supplemented with 1× PIC, 2 mg/ml lysozyme and 0.2 mg/ml lysostaphin (Sigma, St. Louis, Mo.), and incubated at 37° C. for three hours. The tube was centrifuged for 5 min at 20800 ×g and the supernatant was collected. Proteins were separated by electrophoresis in a 10% Novex Bis-Tris gel (Invitrogen, Carlsbad, Calif.), and transferred onto PVDF membrane (Invitrogen, Carlsbad, Calif.). The membrane was incubated with PBS containing 0.05% Tween-20 (PBS-T) and 5% milk for 1 hour at room temperature. Mouse anti-SdrX hyperimmune serum was added at a 1:200 dilution. For competition, 250 μg of rSdrX-A was added to the SdrX antibody. The membrane was incubated overnight at 4° C., washed three times with PBS-T, and incubated with the HRP-conjugated goat anti-mouse antibody at a 1:5000 dilution for 1 hour at room temperature. After washing three times in PBS-T, the membrane was incubated with SuperSignal West Pico Chemiluminescent substrate (Pierce, Rockford, Ill.) for 5 min., and exposed to X-ray film.

rSdrX-A Domain Ligand Binding. 96-well Costar EIA plates (Corning Incorporated, Corning N.Y.) were coated overnight at 2-8° C. with 0.25 μg/well rSdrX-A in 1× PBS (pH 7.4). At the end of the incubation, the plates were washed 4 times with buffer containing 1× PBS (pH 7.4) and 0.05% Tween 20, and blocked with 1% BSA for one hour at room temperature. After washing with buffer containing 1× PBS (pH 7.4) and 0.05% Tween 20, the plates were incubated with human collagen type VI (Rockland Immunochemicals, Gilbertsville, Pa.), collagen type I (Cohesion, Palo Alto, Calif.), human fibrinogen (Enzyme Research Laboratories, South Bend, Ind.) for 1 hour at room temperature. Following the incubation, unbound protein was removed by washing with buffer containing 1× PBS (pH 7.4) and 0.05% Tween 20. A 1:2,000 dilution of biotin conjugated rabbit anti-Collagen VI (Abcam Ltd, Cambridge, UK), 1:2,000 dilution of biotin conjugated affinity purified rabbit anti-Collagen I (Rockland Immunochemicals, Gilbertsville, Pa.), 1:4,000 dilution of Horse Radish Peroxidase (HRP) conjugated goat anti-Fibrinogen (Abeam Ltd, Cambridge, UK) detection antibodies were added to the wells containing the corresponding human protein. The plates were incubated for one hour at room temperature. Unbound detection antibodies were removed by washing with buffer containing 1× PBS (pH 7.4) and 0.05% Tween 20. The reactions were developed using a 2,2′-azino-di (3-ethylbenzthiazoline-6-sulfonate) (ABTS)-H₂O₂ substrate system (KPL, Gaithersburg, Md.) (100 μl/well, 10 min at room temperature) and the absorbance was read at 405 nm using a Spectra-MAX 190 plate reader (Molecular Devices Corporation, Sunnyvale, Calif.).

Bacterial Adherence Assay. Adherence assays were performed as previously described (8) with modifications. 1:100 dilution of an overnight culture of S. capitis 35661 was incubated for 4 hours at 37° C. in Tryptic Soy Broth with agitation (250 rpm). The early log phase cultures were centrifuged at 960 g for 10 minutes at 4° C. The bacterial pellet was washed twice and resuspended in 20 ml 1× PBS (Life Technologies, Inc., Rockville, Md.). After washing, the bacterial suspension was adjusted to an OD₆₀₀ₙₘ of 2.9 in 1× PBS. Costar ELISA plates (Corning Incorporated, Corning, N.Y.) were coated with 100 μl/well of 2 μg/ml human collagen type VI (Rockland Immunochemicals, Gilbertsville, Pa.) or Human Fibrinogen (Enzyme Research Laboratories, South Bend, Ind.) overnight at 4° C. The coated plates were washed 3 times with 200 μl/well 1× PBS buffer. Plates were blocked with 1% BSA, 200 μl/well for one hour at room temperature and then washed. Serial dilutions of protein A affinity purified rabbit anti-SdrX A domain hyperimmune serum or normal rabbit IgG in 1× PBS were mixed with an equal volume of S. capitis cells and incubated for one hour at room temperature. The antibody/bacteria mixtures were added to the plates and incubated for two hours at 37° C. The plates were washed to remove non-adherent bacteria. The adherent bacteria were fixed with 25% formaldehyde at room temperature for thirty minutes. The formaldehyde was removed by washing and the adherent bacteria were stained with 0.5% crystal violet (100 μl/well) for one minute at room temperature. Excess crystal violet was removed by washing the plate 4 times with 1× PBS (200 μl/well). In order to dissolve the crystal violet, the plate was incubated for 15 minutes at room temperature with 5% acetic acid solution (100 μl/well). The absorbance was read at 570 nm using a Spectra MAX 190 plate reader (Molecular Devices Corporation, Sunnyvale, Calif.).

Nucleotide sequence accession number. The nucleotide sequence of the sdrX gene has been deposited in the GenBank database under accession number AY510088.

Results

Cloning and sequencing sdrX. Genomic DNA was isolated from the S. epidermidis strain K28. This DNA was used as a template for PCR to generate a 791 base pair gene fragment spanning the B1 through R region of the sdrG gene (FIG. 1A). This PCR product was used as a hybridization probe (R region probe) to determine if the S. capitis genome contained genes which shared sequence similarities with Sdr family proteins. A genomic Southern blot of DNA prepared from S. capitis strain 49326 was probed with the sdrG R region fragment using low stringency hybridization. Two Hind III fragments approximately 5.5 Kb and 4.5 Kb hybridized with the probe (FIG. 1B). In order to isolate and identify these potential Sdr family gene sequences, a mini-genomic library was constructed by digesting genomic DNA from S. capitis strain 49326 with HindIII and cloning 3 to 6 Kb size fractionated fragments into the pUC18 vector. The inserts of 5.5 Kb and the 4.5 Kb were subsequently cloned by colony hybridization and were sequenced.

The 5.5 Kb insert contained an open reading frame (ORF) of 2136 base pairs encoding a protein of 711 amino acids with calculated molecular weight of 76.71 kDa. The deduced protein sequence (FIG. 1C) included a long serine-aspartate repeat (Sdr) region characteristic of the Sdr protein family. In keeping with current naming conventions, the newly identified protein was named SdrX. SdrX contained other protein sequence features typical of other Sdr proteins (18) (FIG. 2). The N-terminal portion of the encoded protein contained a putative signal peptide of 39 amino acids as predicted by SignalP software (http://www.cbs.dtu.dk/services/SignalP). Following the signal sequence, there was a 214 amino acid region, we have designated as the putative A domain. Interestingly, the A domain of SdrX has little sequence similarity to previously described Sdr proteins (FIG. 2) but does share 47% similarity to the Aas protein, a fibronectin-binding autolysin from S. saprophyticus (10) and 44% similarity to the AtIC protein from S. caprae (1). The A region was followed by a B region containing a series of short tandem repeats (FIG. 1C). Further downstream, followed a highly repetitive region of 206 amino acid residues, composed of tandemly repeated serine-aspartate residues with other residues such as cysteine, glutamic acid, and glycine found sporadically through the region. The C-terminal portion of the protein contained the sequence LPDTG which corresponds to the cell-wall-anchoring motif LPXTG found in all Sdr family proteins (18). A membrane-spanning region, and a positively charged tail, features also common to most Sdr family proteins were identified in the C-terminus of the protein sequence (FIG. 1C).

Prevalence of the sdrX gene in S. capids strains. Genomic DNAs from 9 different ATCC strains of S. capitis and two clinical isolates of S. capitis were amplified by PCR using primers specific for the sdrX gene. All of the strains tested were found to contain the sdrx gene (data not shown).

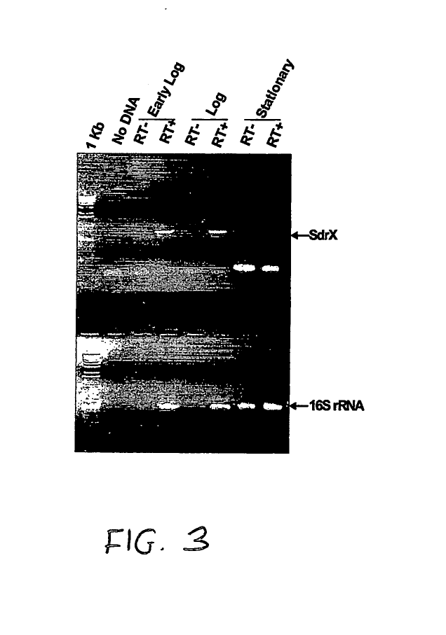

Transcription of sdrX at different stages of bacterial growth. To determine if sdrX gene transcription is regulated during the growth cycle, total RNA was isolated from S. capitis 49326 cultures at early log, log and stationary phases. RT-PCR was performed using primers specific for sdrX. RT-PCR using primers specific for 16S rRNA was also performed as a control. As shown in FIG. 3, the sdrX gene was transcribed at both early log and log phases, but sdrX specific RNA could not be detected at stationary phase, indicating that the transcription of sdrX is regulated during the different stages of bacterial growth.

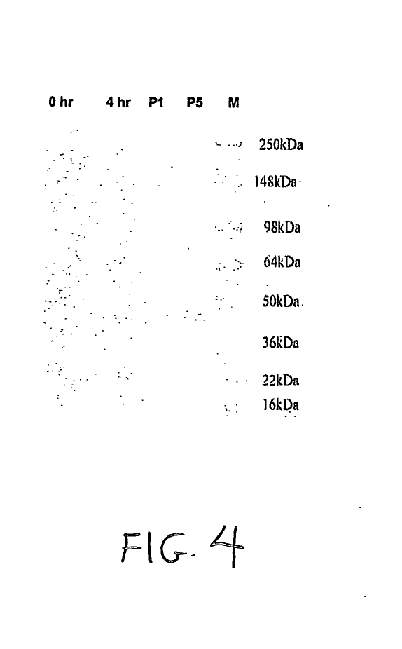

Expression and purification of the A domain of SdrX. The A domain of SdrX was cloned into pQE-30 vector and expressed as a 6× His-tagged fusion protein in the E. coli M15[pREP4] strain. One prominent 45 kDa band was detected in the cell extract after induction for 4 hours with 1 mM IPTG (FIG. 4). This band was not present in the absence of induction. Although the apparent molecular weight of the protein by SDS-PAGE was significantly different from the predicted molecular weight for the recombinant protein, mass spectroscopy analysis confirmed a molecular weight of 25.7 kDa. The migration pattern of the recombinant SdrX A domain (rSdrX-A) in SDS/PAGE is consistent with other recombinant A domains of previously identified MSCRAMM® proteins (our own observation).

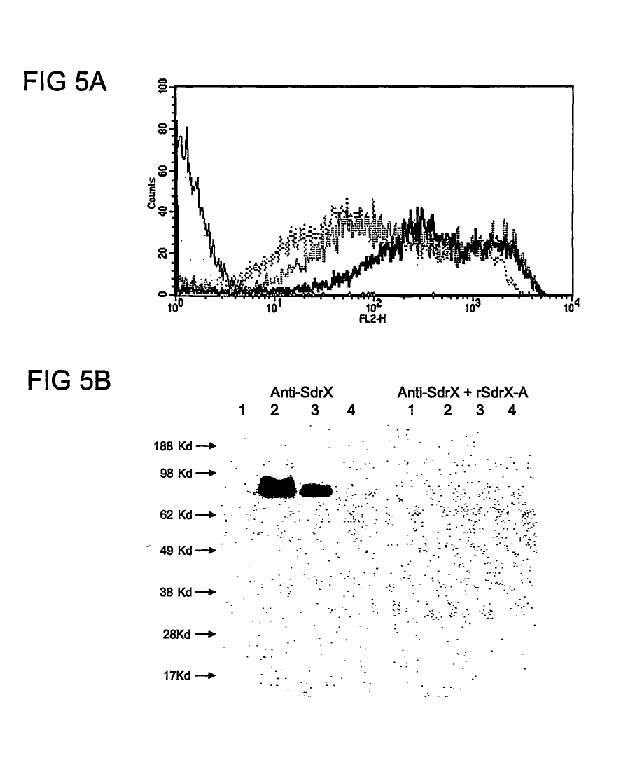

Localization of SdrX Expression. The presence of a cell wall anchoring motif, LPDTG, in the cell wall spanning domain of SdrX suggested that the protein was likely to be associated with the cell wall (27). To determine if SdrX is indeed expressed on the surface of S. capitis, a panel of strains was analyzed by flow cytometry using an rSdrX-A specific antiserum. The anti-rSdrX-A serum recognized the surface of all of the strains tested. Strain 35661 was found to have the highest level of antibody recognition (data not shown). This strain was selected for a time course experiment to determine if SdrX protein expression was influenced by culture conditions. S. capitis cultures were analyzed at early log, log and stationary growth phases by flow cytometry (FIG. 5A). The highest mean fluorescence was observed with early log phase cultures. Analysis of later stages of growth resulted in lesser immunofluorescence indicating lower levels of antigen expression. This finding was corroborated by Western immunoblotting analysis. Cytoplasmic and cell wall protein fractions were prepared from early log, log and stationary phase of S. capitis cultures. A band of 80 Kda was detected in the cell wall fractions at all time points. However, signal intensity was highest in early log and log phase culture samples (FIG. 5B). The signal from the 80 Kda band was completely eliminated when the Western blot was carried out in the presence of soluble rSdrX-A, indicating that the antibody signal was specific for SdrX. SdrX protein was not observed in the cytoplasmic fraction, suggesting that the vast majority of SdrX protein was cell wall associated.

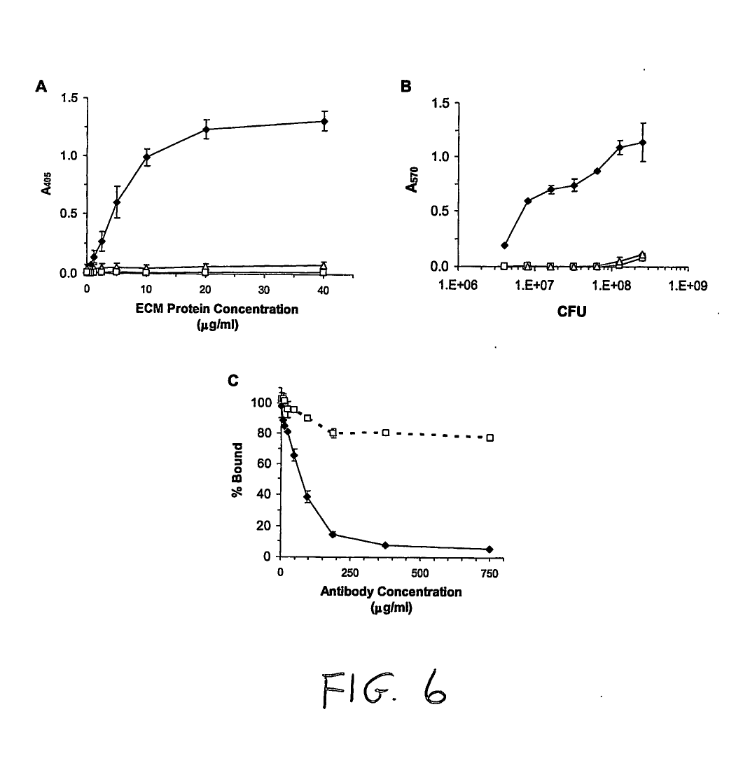

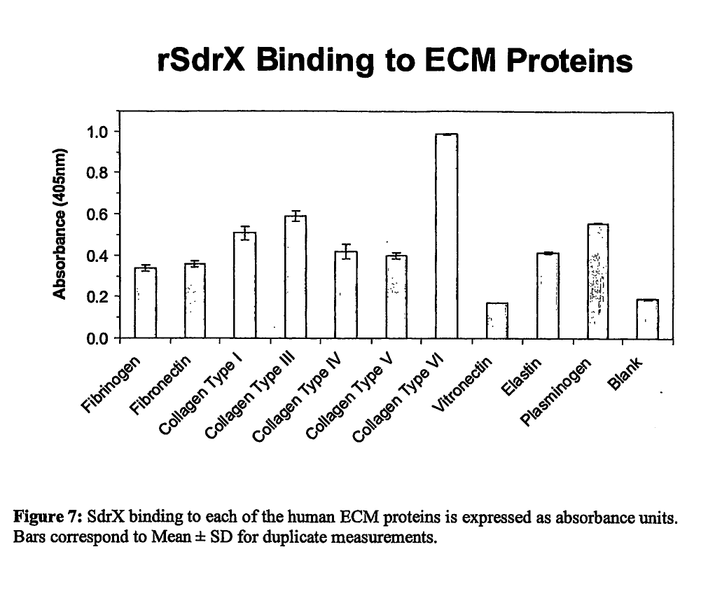

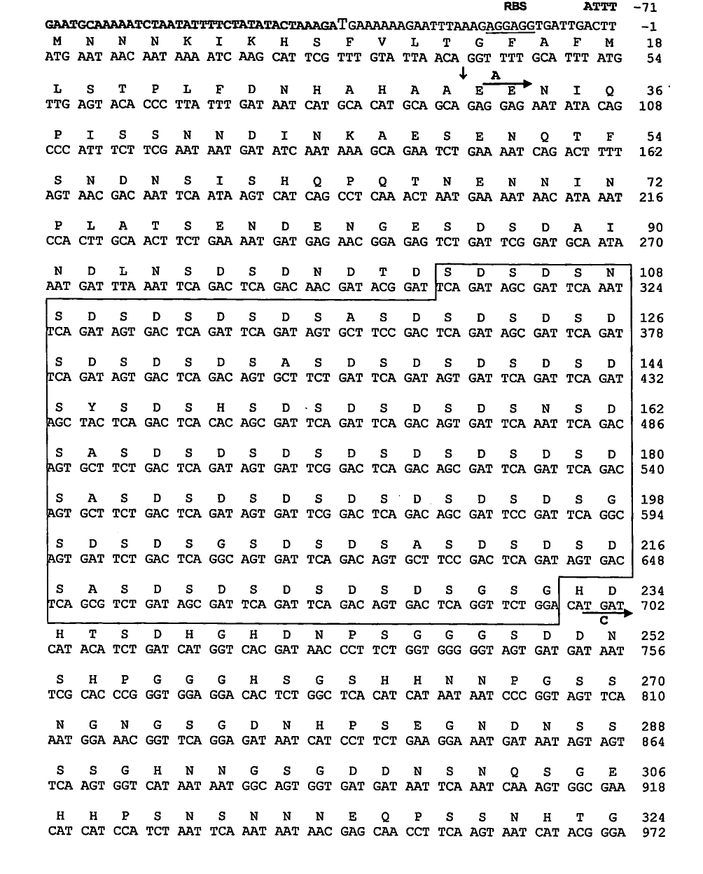

Identification of the rSdrX-A ligand. To identify potential ligands for SdrX, human extracellular matrix proteins were tested for their ability to bind rSdrX-A in an ELISA assay. The proteins evaluated included human collagen types I, III, IV, V and VI, fibrinogen, fibronectin, plasminogen, vitronectin and elastin. The preliminary result from the initial screening showed that human collagen type VI had significant binding activity to rSdrX-A (data not shown). Therefore, human collagen type VI was subsequently used for a detailed study. FIG. 6A showed that the binding of SdrX A-domain to collagen VI was specific and dose-dependent.

S. capitis binding to human type VI collagen. Log phase cultures of S. capitis strain 35661 were incubated on microtiter plates coated with human collagen type VI, human fibrinogen or BSA (FIG. 6B). S. capitis bound specifically to collagen type VI coated plates and increased as a function of bacterial concentration. To determine if SdrX was responsible for the collagen type VI binding activity observed in S. capitis, the bacteria were pre-incubated with increasing concentrations of rabbit anti-SdrX-A polyclonal antibody prior to performing the adherence assay (FIG. 6C). The anti-SdrX antibody was able to inhibit S. capitis binding to collagen type VI by as much as 95% whereas equal concentrations of normal rabbit IgG had little effect. This result suggests that most if not all of the collagen type VI binding activity observed in S. capitis was mediated by SdrX.

Discussion

A molecular biology approach was used to determine if Sdr family proteins exist in S. capitis. A DNA fragment corresponding to the B and R regions of the sdrG gene was used to probe the S. capitis genome. A novel member of the Sdr family of MSCRAMM®s was identified, cloned and sequenced. The deduced protein sequence was compared to the published protein sequences of other Sdr family molecules. The overall structure of the coding region was found to follow the general pattern observed in other Sdr family proteins (18) and included a signal sequence, an A domain, a repetitive domain termed BX, an SD repeat region, a cell wall anchor region with an LPXTG motif sequence (LPDTG amino acids 674-678), a hydrophobic membrane spanning region and a series of postively charged residues at the c-terminus. Individual domains of SdrX were compared to other members of the Sdr family using Clustal W analysis.

Comparison of the SdrX signal sequence showed the greatest homology (˜52%) with SdrC, SdrD, and SdrF. The A domain of SdrX was compared to other Sdr protein sequences and showed little or no homology (less than or equal to 11%). The A domain sequence was also used to perform a BLAST search of the public database at NCBI. Only two protein sequences were found to have homologies greater than 40%, the AtIC protein (44% homology) from S. caprae (1) and the Aas protein (47% homology) a fibronectin-binding autolysin of S. saprophyticus (10). The nature and extent of the relationship of SdrX to either of these proteins is currently not known. Although a conserved sequence (TYTFTDYVD) has been reported to be in the A domains of all S. aureus Sdr proteins (12, 22) and in the S. epidermidis proteins SdrF and SdrG (18), this sequence was not present in the SdrX A-domain. The absence of this sequence suggests that the A-domain of SdrX appears to be of unique structure and unrelated to previously described Sdr protein A-domains.

The repetitive region of 163 amino acids found between the A domain and the SD dipeptide repeat region in SdrX is made up of short repeated sequences varying in length. The repeats are high in S and D content (56% SD overall) but are sufficiently divergent from the dipeptide repeat R region to be categorized as a separate domain. This sequence is considerably divergent from the B regions described in other Sdr proteins. This region in SdrX was therefore named BX to distinguish it from previously described B regions.

The R region of SdrX is typical in size (206 amino acids) for R regions found throughout the Sdr protein family. The presence of this domain places SdrX unequivocally in the Sdr family of staphylococcal proteins.

All of the Sdr proteins identified to date also include a conserved sequence motif, LPXTG (18). This sequence is a substrate for sortase, a transpeptidase that cleaves and covalently links the protein to peptidoglycan in the cell wall, allowing for surface expression of the molecule (17, 28). The sequence LPDTG is found in SdrX at position 674. Taken together, the R region and the BX region provide 419 amino acids between the end of the putative A domain and the LPDTG cell wall anchoring motif. For ClfA, it has been reported that the R region must be 80 residues in length (112 residues in total from A domain to LPXTG) to support wild-type clumping function (9). Therefore, the R region of SdrX would appear to be of sufficient length to allow for exposure of the A domain on the surface of S. capitis. Two lines of evidence demonstrate that SdrX is indeed expressed on the cell surface. By Western blot analysis it was shown that SdrX is present in cell wall fractions from S. capitis but not in cytoplasmic preparations of the same cultures. Also, the A domain of SdrX was found to be accessible to antibodies on the surface of viable S. capitis as measured by flow cytometry. The available data therefore demonstrates that the SdrX protein is a surface expressed protein, as predicted from the primary sequence.

C-terminal to the LPDTG sequence is a 23 amino acid sequence containing a high percentage of hydrophobic residues (57%) followed by a short highly polar sequence. Similar regions have been described previously for Sdr proteins (18) and are thought to function as a membrane spanning region and a cytoplasmic tail. Overall, the analysis of the SdrX protein sequence has led to the conclusion that SdrX is a novel member of the Sdr gene family and the first such protein described in S. capitis.

Beyond the primary sequence data, our investigations of SdrX have focused on the distribution and expression of SdrX. Genomic PCR was carried out to determine if the sdrX gene is widely distributed among S. capitis strains. A panel of eleven different strains was analyzed. All of these strains were positive in the PCR assay for the presence of the sdrX gene. Although the panel was relatively small, it included both commonly used laboratory strains as well as two unique clinical isolates collected in a NICU. The existing data would anticipate that the sdrX gene will be found to be widely distributed among S. capitis strains.

SdrX expression was analyzed at both the RNA and protein level. Results of RT-PCR demonstrated that the sdrX transcript could be found in early and mid log phase cultures but not in stationary phase. Protein expression data collected by Western blofting and flow cytometry were in agreement with the RT-PCR data in that the greatest protein signals were obtained in early log phase and the lowest levels were in bacteria grown to stationary phase. The finding that RNA transcription is no longer occurring in stationary phase cultures, but protein is still detectable, suggests that the protein remains on the surface of the bacteria in the absence of de novo synthesis and may indicate that the protein is a relatively stable and long lived molecule. The recombinant protein rSdrX-A was used to screen ECM proteins for potential ligands by ELISA. rSdrX-A was shown to specifically bind collagen type VI. Based on this finding, we evaluated the adherence of viable S. capitis cells to microtiter plates coated with collagen type VI. Adherence of the bacteria to collagen type VI was demonstrated. Moreover, antibodies generated against the A domain of SdrX were capable of nearly complete abrogation of this binding. Therefore, the data suggests that SdrX is principally responsible for the collagen type VI binding activity of S. capitis strain 35661. The collagen type VI monomer is made up of three different alpha chains, each of which consists of a short helical region separating globular domains at the N- and C-termini (5). The molecule is secreted as a tetramer which then assembles in the extracellular matrix (ECM) to form microfibrils (6). Collagen type VI has been reported to bind to a wide range of molecules including other collagens (types I, II, and IV), decorin, NG2, and integrins (α1β1 and α2β1) (2, 3, 4, 15, 24). Collagen type VI is found in the ECM of virtually all connective tissues including skin, bone, cartilage, nerves, cornea and skeletal muscle (14, 15). The microfibrillar meshwork formed by collagen VI plays an important role in cell attachment. Indeed, mutations in collagen type VI genes have been linked to an inherited muscular dystrophy, Bethlem myopathy (11, 16, 23). The ubiquitous expression of collagen type VI makes this molecule and ideal ligand for bacterial adherence. Whether or not collagen type VI binding by S. capitis plays a role in pathogenesis remains to be determined, but it would appear that SdrX would be able to be utilized in the development of antibody therapies against S. capitis infection in light of the fact that antibodies can be generated against it have been shown to inhibit the activity of SdrX.

### Example 2

Identifying and Isolating the SdrZL Protein

Overview

The nucleotide sequences in S. capitis 49326 hybridizing at low stringency with the repeat region probe (FIG. 1a) from sdrG of S. epidermidis K28 were found on two HindIII fragments about 5.5 Kb and 4.5 Kb (FIG. 1 ). A mini-genomic library was constructed by cloning HindIII digested DNA fragments of 3 to 6 Kb into pUC18 vector. The inserts of 5.5 Kb and the 4.5 Kb were subsequently cloned by colony hybridization and were sequenced. The sequencing of the 5.5 Kb insert, identified as SdrX, is described above in Example 1.

Materials and Methods

Reagents. Restriction enzymes, calf intestinal alkaline phosphatase, and T4 DNA ligase were purchased from New England Biolabs (Beverly, Mass.). The Taq DNA polymerase, and dNTPs are from GIBCO-BRL (Rockville, Md.). G/Nome DNA kit is purchased from Bio 101 (Carlsbad, Calif.) and used for genomic DNA isolation. Lysozyme and lysostaphin are from Sigma (St. Louis, Mo). The Zeta-probe GT genomic Blotting and C/P lift membranes are purchased from Bio-Rad (Hercules, Calif.) for Southern and colony hybridizations respectively. The supersignal West Pico Chemiluminescent Substrate is from PIERCE (Rockford, Ill.), and Anti-Digoxigenin-POD Fab fragment was from Roche (Indianapolis, Ind.).

Bacterial strains and plasmids. Escherichia coli strain XL10-Gold ultra-competent cell was purchased from Stratagene (La Jolla, Calif.) and used as host for DNA transformation. Plasmid pUC18 was used for cloning. Chromosomal DNA was prepared from S. epidermidis K28, S. capitis 49326, S. haemolyticus, S. hominis, S. simulans, and S. warneri

Molecular Biology techniques. Genomic DNA isolation from bacteria: The bacterial cells were cultured in 5 ml of LB broth for overnight at 37° C. The overnight culture (5 ml) was then inoculated into 50 ml of the LB broth, and cultured at 37° C. for 4 hrs. Bacterial cells were collected by centrifugation at 4000 rpm (Rotor SS34, Beckman) for 10 minutes. The cell pellet was resuspended in 1.8 ml of the “Cell resuspension” solution (G/Nome DNA kit, Bio 101) plus 36 ul of lysozyme (100 mg/ml) and 20 ul of lysostaphin (10 mg/ml), and was incubated at 37° C. for 2 hrs. 50 ul of the RNase Mixx and 100 ul of the “Lysis solution” (G/Nome DNA kit, Bio 101) were added to the tube containing the bacterial cells. The mixture was incubated at 55° C. for 15 minutes. 30 ul of the Proteinase Mixx (G/Nome DNA kit, Bio 101) was added and incubated at 55° C. for 3 hrs. 500 ul of the “Salt out” solution (G/Nome DNA kit, Bio 101) was added and incubated at 4° C. for 10 minutes. The contents were divided into 2 eppendorf tubes and centrifuged the at top speed in a bench top microcentrifuge at 4° C. for 15 minutes. The supernatant was collected into a 50 ml centrifuge tube, and 2 ml of TE, and 8 ml of cold 100% ethanol were added. The tube was centrifuged immediately at 13000 rpm (Rotor-SS34, Beckman) for 20 minutes. The DNA pellet was washed with 70% ethanol, and the DNA was air dried. The DNA was dissolved in 500 ul of sterile water.

Digoxigenin-labelled probe: The DNA sequence including the B and R regions from SdrG was amplified by PCR in the presence of the PCR DIG Probe Synthesis mix (Roche), the forward primer, 5′-CCGCTTAGTMTGTATTG-3′ and the reverse primer, 5′-TCTTATCTGAGCTATTG-3′, the genomic DNA template from S. epidermidis K28, and the Taq DNA polymerase (Gibco-BRL). The PCR was conducted at; 94° C., 40 seconds, 42° C., 40 seconds, 72° C., 1 min for 30 cycles. The PCR product was purified using the QiAquick Gel Extraction Kit (Qiagen).

Southern hybridization: About 1 μg of genomic DNA was digested with 20 U of HindIII at 37° C. overnight and separated in a 0.8% agarose gel. Before transfer, the gel was soaked in 0.25 N HCl for 15 minutes at room temperature and rinsed in water twice. The DNA fragments were transferred onto the Zeta-probe membrane in 0.4 N NaOH by capillary action for 4 hrs. After transfer, the membrane was baked in a hybridization oven at 80° C. for 1 hr. The membrane was incubated in pre-hybridization solution (0.25 M sodium phosphate, pH 7.2 and 7% SDS) for 30 minutes at 45° C. in a hybridization oven. Hybridization was performed in the pre-hybridization solution containing the addition of the Digoxigenin-labelled probe (denatured by boiling for 10 minutes) at 45° C. overnight. Following the completion of hybridization, the membrane was washed 4 times in 20 mM sodium phosphate, pH 7.2 and 5% SDS for 15 minutes at 45° C., followed by washing 4 times in 20 mM sodium phosphate, pH 7.2 and 1% SDS for 15 minutes at 45° C. After washing, the membrane was briefly rinsed in PBS-Tween and incubated in 5% milk in PBS-Tween for 1 hr at room temperature. The membrane was incubated in 5% milk in PBS-Tween plus the anti-Digoxigenin-POD antibody (1/1000 dilution) for 1 hr at room temperature. The membrane was washed 3 times in PBS-Tween for 5 minutes. The membrane was then incubated in the supersignal West Pico Chemiluminescent Substrate for 5 minutes at room temperature. The membrane was then exposed to X-ray film.

Colony Hybridization: Bacterial plates were chilled at 4° C. for 30 min. The plates were covered with a 85 mm C/P Lift Membrane (BioRad) till it was wet. Holes were punched at 11 clock, 5 clock, and 9 clock through the membrane and the agar plate. The positions of the alignment holes were marked on the bottom of the plate. The membrane was lifted, and put on top of Whatmann paper soaked with the Lysis-denaturing solution (0.5N NaOH+1% SDS) for 10 min. The membrane was washed with 2×SSC and baked at 80° C. for at 30 min. Pre-hybridization and hybridization were done under the same conditions as for the Southern hybridization. If multiple lifts were placed in one tube, the membranes were separated from each other with a “Hybridization Mesh” (Fisher, Pittsburgh, Pa.).

Results

Within the 4.5 Kb insert as described above and shown in FIG. 1B, DNA sequences encoding another Sdr-like protein were found. The primary sequence and the structural organization of the encoded protein are similar to that of SdrZ from S caprae 96007 (accession number AF048595) and SdrH from S. epidermidis 9491 (accession number AF245043). Therefore, the newly identified gene is called SdrZL (stands for SdrZ-like). The N-terminal part of the deduced amino acid sequence contains a putative signal peptide of 30 amino acid residues with 77% similarity to the signal sequence of SdrZ. A short A domain 60 amino acids in length shares 52% similarity to the A domain of SdrZ. Following the A region, a stretch of 130 amino acid residues consisting of a serine-aspartate dipeptide repeat was found. A, G, H, N, and Y residues were also found within the SD repeat. The SD repeat region was followed by a C region of 333 amino acid residues. The C region shares 62.9% similarity to that of SdrZ (FIG. 8a,b). Upstream of the SdrZL gene are DNA sequences that match the AgrB (Accessory gene regulator) gene of S. capitis strain CCM2734 (Dufour, P et al., 2002).

The following journal articles referred to above are herein incorporated by reference as if set forth in their entirety herein:
1. Allignet, J., S. Aubert, K. G. Dyke, and N. El Solh. 2001. Staphylococcus caprae strains carry determinants known to be involved in pathogenicity: A gene encoding an autolysin-binding fibronectin and the ica operon involved in biofilm formation. Infect. Immun. 69 (2): 712-718.
2. Bidanset, D. J., C. Guidry, L. C. Rosenberg, H. U. Choi, R. Timpi and M. Hook. 1992. Biding of the proteoglycan decorin to collagen type VI. J. Biol. Chem. 267 (8): 5250-5256.
3. Bonaldo, P., V. Russo, F. Bucciotti, R. Doliana and A. Colombatti. 1990. Structural and functional features of the alpha 3 chain indicate a bridging role for chicken collagen VI in connective tissues. Biochemistry 29 (5): 1245-1254.
4. Burg, M. A., E. Tillet, R. Timpi and W. B. Stallcup. 1996. Binding of the NG2 proteoglycan to type VI collagen and other extrcellular matrix molecules. J. Biol. Chem. 271 (42): 26110-26116.
5. Engel J., H. Furthmayr, E. Odermatt, H. von der Mark, M. Aumailley, R. Fleischmajer, R. Timpl. 1985. Structure and macromolecular organization of type VI collagen. Ann N Y Acad Sci. 460: 25-37.
6. Furthmayr, H., H. Wiedemann, R. Timpl, E. Odermatt and J. Engel. 1983. Electon-microscopical approach to a structural model of intima collagen. Biochem. J. 211 (2): 303-311.
7. Hall, A. E., P. J. Domanski, P. R. Patel, J. H. Vernachio, P. J. Syribeys, E. L. Gorovits, M. A. Johnson, J. M. Ross, J. T. Hutchins, and J. M. Patti. 2003. Characterization of a Protective Monoclonal Antibody Recognizing Satphylococcus aureus MSCRAMM Protein Clumping Factor A. Infect. Immun. 71(12): 6864-6870.
8. Hartford, O., L. O'Brien, K. Schofield, J. Wells and T. J. Foster. 2001. The Fbe (SdrG) protein of Staphylococcus epidermidis HB promotes bacterial adherence to fibrinogen. Microbiology. 147:2545-2552.
9. Hartford, O., P. Francois, P. Vaudaux, and T. J. Foster. 1997. The dipeptide repeat region of the fibrinogen-binding protein (clumping factor) is required for functional expression of the fibrinogen-binding domain on the Staphylococcus aureus cell surface. Mol. Microbiol 25(6): 1065-1076.
10. Hell, W., H. G. Meyer, S. G. Gatermann. 1998. Cloning of aas, a gene encoding a Staphylococcus saprophyticus surface protein with adhesive and autolytic properties. Mol. Microbiol. 29 (3): 871-881.
11. Jobsis G. J., H. Keizers, J. P. Vreijling, M. de Visser, M. C. Speer, R. A. Wolterman, F. Baas and P. A. Bolhuis. 1996. Type VI collagen mutations in Bethlem myopathy, an autosomal dominant myopathy with contractures. Nat. Genet. 14 (1): 113-115.
12. Josefsson, E., K. W. McCrea, D. Ni Eidhin, D. O'Connell, J. Cox, M. Hook, and T. J. Foster. 1998. Three new members of the serine-aspartate repeat protein multigene family of Staphylococcus aureus. Microbiology 144 (Pt 12):3387-95.
13. Josefsson, E., O. Hartford, L. O'Brien, J. M. Patti, and T. Foster. 2001. Protection against experimental Staphylococcus aureus arthritis by vaccination with clumping factor A, a novel virulence determinant. J. Infect. Dis. 184:1572-80.
14. Keene, D. R., E. Engvall and R. W. Glanville. 1995. Ultrastructure of type VI collagen in human skin and cartilage suggests an anchoring function for this filamentous network. J. Cell Biol. 107: 1995-2006.
15. Kuo, H-J. C. L. Maslen, D. R. Keene and R. W. Glanville. 1997. Type VI collagen anchors endothelial basement membranes by interacting with type IV collagen. J. Biol. Chem. 272 (42): 26522-26529.
16. Lamande, S. R., J. F. Bateman, W. Hutchison, R. J. McKinlay Gardner, S. P. Bower, E. Byrne and H-H. M. Dahl. 1998. Reduced collagen VI causes Bethlem myopathy: a heterozygous COL6A1 nonsense mutation results I mRNA decay and functional haploinsufficiency. Human Molecular Genetics 7 (6): 981-989.
17. Mazmanian, S. K., G. Liu, H. Ton-That, O. Schneewind. 1999. Staphylococcus aureus Sortase, an enzyme that anchors surface proteins to the cell wall. Science 285: 760-763.
18. McCrea, K W., O. Hartford, S. Davis, D. N. Eidhin, G. Lina, P. Speziale, T. J. Foster, M. Hook. 2000. The serine-aspartate repeat (Sdr) protein family in Staphylococcus epidermidis. Microbiology 146 (7):1535-46.
19. McDevitt, D., P. Francois, P. Vaudaux, and T. J. Foster. 1994. Molecular characterization of the clumping factor (fibrinogen receptor) of Staphylococcus aureus. Mol. Micro. 11:237-248.
20. McDevitt, D., P. Vaudaux, and T. J. Foster. 1992. Genetic evidence that bound coagulase of Staphylococcus aureus is not clumping factor. Infect. Immun. 60:1514-1523.
21. Ni Eidhin, D., S. Perkins, P. Francois, P. Vaudaux, M. Hook, and T. J. Foster. 1998. Clumping factor B (ClfB), a new surface-located fibrinogen-binding adhesin of Staphylococcus aureus. Mol. Microbiol. 30:245-57.
22. O'Connell, D. P., T. Nanavaty, D. McDevitt, S. Gurusiddappa, M. Hook and T. Foster. 1998. The fibrinogen-binding MSCRAMM (Clumping Factor) of Staphylococcus aureus has a Ca2+-dependent inhibitory site. J. Biol. Chem. 273(12): 6821-6829.
23. Pan, T-C., R-Z. Zhang, M. A. Periicak-Vance, R. Tandan, T. Fries, J. Stajich, K. Viles, J. M. Vance, M-L. Chu and M. C. Speer. 1998. Missense mutation in a von Willebrand factor type A domain of the α3(VI) collagen gene (COL6A3) in a family with Bethlem myopathy. Human Molecular Genetics 7 (5): 807-812.
24. Pfaff, M., M. Aumailley, U. Specks, J. Knolle, H. G. Zerwes and R. Timpl. 1993. Integrin and Arg-Gly-Asp dependence of cell adhesion to the native and unfolded triple helix of collagen type VI. Exp. Cell Res. 206 (1): 167-176.
25. Patti, J. M., and M. Hook. 1994a Microbial adhesins recognizing extracellular matrix macromolecules. Curr Opin Cell Biol. 6(5):752-758.
26. Patti, J. M., B. A. Aleen, M. J. McGavin, and M. Hook. 1994b. MSCRAMM-mediated adherence of microorganisms to host tissues. Annu. Rev. Microbiol. 45: 585-617.
27. Schneewind, O., D. Mihaylova-Petkov, and P. Model. 1993. Cell wall sorting signals in surface proteins of S. capitis bacteria. EMBO J. 12: 4803-4811.
28. Schneewind, O., A. Fowler, and K. F. Faull. 1995. Structure of the cell wall anchor of surface proteins in Staphylococcus aureus. Science 268: 103-106.
29. Stoll, B. J., N. Hansen, A A. Fanaroff, L L. Wright, W A. Carlo, R A. Ehrenkranz, J A. Lemons, E F. Donovan, A R. Stark, J E. Tyson, W. Oh, C R. Bauer, S B. Korones, S. Shankaran, A R. Laptook, D K. Stevenson, L A. Papile, W K. Poole. 2002. Late-onset sepsis in very low birth weight neonates: the experience of the NICHD Neonatal Research Network. Pediatrics. 110:285-91.
30. Van Der Zwet, W. C., Y. J. Debets-Ossenkopp, E. Reinders, M. Kapi, P. H. M. Savelkoul, R. M. Van Elburg, K. Hiramatsu and C. M. J. E. Vandenbroucke-Grauls. 2002. Nosocomial Spread of a Staphylococcus capitis Strain with Heteroresistance to Vancomycin in a Neonatal Intensive Care Unit. J. Clin. Microbiol. 40 (7): 2520-2525.
31. Vernachio, J., A. S. Bayer, T. Le, Y.-L. Chai, B. Prater, A. Scnhneider, B. Ames, P. Syribeys, J. Robbins, and J. M. Patti. 2003. Anti-Clumping Factor A Immunoglobulin Reduces the Duraiton of Methicillin-Resistant Staphylococcus aureus Bacteremia in an Experimental Model of Infective Endocarditis. Antimicrob. Agents Chemother. 47(11): 3400-3406.
32. Dufour, P., Jarraud, S., Vandenesch, F., Greenland, T., Novick R P., Bes, M., Etienne, J. and Lina, G. High Genetic Variability of the agr Locus in Staphylococcus Species. J. Bacteriol. 184: 1180-1186. 2002.

What is claimed is:

1. An isolated surface serine-aspartate repeat (Sdr) protein from Staphylococcus capitis.

2. The isolated surface Sdr protein of claim 1 wherein the protein is selected from the group consisting of the SdrX protein and the SdrZL protein.

3. The isolated surface protein of claim 2 wherein the protein is selected from the group consisting of the A domain of SdrX and the A domain of SdrZL.

4. The isolated surface protein of claim 1 having the sequence of SEQ ID NO: 2.

5. The isolated surface Sdr protein of claim 1 wherein the protein is encoded by a nucleic acid selected from the group consisting of sdrX and sdrZL.

6. The isolated surface Sdr protein of claim 1 wherein the protein has the ability to bind collagen VI.

7. A pharmaceutical composition comprising the isolated protein of claim 1 and a pharmaceutically acceptable vehicle carrier or excipient.

8. An isolated antibody that can recognize the protein of claim 1.

9. A therapeutic composition for treating or preventing a staphylococcal infection comprising an antibody according to claim 8 in an amount effective to treat or prevent a staphylococcal infection in a human or animal patient in need of such treatment and a suitable vehicle, excipient or carrier.

10. The antibody according to claim 8 wherein the antibody can recognize the SdrX protein.

11. The antibody according to claim 8 wherein the antibody can recognize the amino acid sequence of SEQ ID NO: 2.

12. The antibody according to claim 8 wherein the antibody can recognize the A domain of the amino acid sequence of SEQ ID NO: 2.

13. A vaccine comprising an immunogenic amount of the protein of claim 1 and a pharmaceutically acceptable vehicle, excipient or carrier.

14. The vaccine of claim 13 wherein the protein is the SdrX protein.

15. Isolated antibody or antisera which can recognize the SdrX protein from S. capitis.

16. The antibody or antisera of claim 15 wherein the protein is the SdrX A domain.

17. An isolated SdrX protein from S. capitis

18. The isolated protein from S. capitis wherein the protein is the SdrX A domain.

19. An isolated nucleic acid coding for the protein of claim 1

20. An isolated nucleic acid coding for the protein having the sequence of SEQ ID NO: 2, or degenerates thereof.

21. The isolated nucleic acid of claim 20 having the sequence of SEQ ID NO: 1.

22. A diagnostic kit for determining the presence of S. capitis Sdr surface proteins in a sample suspected of containing such proteins comprising an antibody according to claim 8, means to introduce the antibody to the sample, and a means for determining the presence of binding of the antibodies and SdrX proteins in the sample.

23. A diagnostic kit for determining the presence of antibodies recognizing SdrX in a sample suspected of containing said antibodies comprising isolated SdrX proteins, means to introduce the proteins to the sample, and a means for determining the presence of binding of the SdrX proteins and the antibodies to SdrX in the sample.

24. A method of treating or preventing an S. capitis infection in a human or animal patient in need of such treatment comprising administering to the patient the isolated protein of claim 1 in an amount effective to treat or prevent an S. capitis infection.

25. A method of treating or preventing an S. capitis infection in a human or animal patient in need of such treatment comprising administering to the patient an antibody according to claim 8 in an amount effective to treat or prevent an S. capitis infection.

26. A method of preventing binding of S. capitis to collagen in a human or animal patient or an in-dwelling medical device comprising administering an antibody according to claim 8 in an amount effective to inhibit binding of S. capitis to collagen.

27. The method of claim 26 wherein the antibody recognizes an SdrX protein.

28. A method of reducing or eliminating an S. capitis infection of an indwelling medical device or implant comprising coating the medical device or implant with a SdrX protein in an amount effective to reduce or eliminate binding of S. capitis to the medical device or implant.

29. The method of claim 28 wherein the medical device is selected from the group consisting of vascular grafts, vascular stents, intravenous catheters, artificial heart valves, and cardiac assist devices.

30. A method of inducing an immunological response to a surface Sdr protein from S. capitis comprising administering to a patient an immunogenic amount of an isolated Sdr surface protein.

31. A purified human donor plasma immunoglobulin composition comprising a purified human donor plasma composition having an antibody titer to an SdrX protein from S. capitis in an amount higher than that found in pooled intravenous immunoglobulin obtained from unselected human donors.

32. The purified human donor plasma immunoglobulin composition of claim 31 wherein said composition is obtained by a method comprising obtaining blood or plasma samples from human donors, screening said samples so as to select those samples having an antibody titer to the SdrX protein from S. capitis in an amount that is higher than that found in pooled intravenous immunoglobulin obtained from unselected donors, recovering blood or plasma from the selected high-titer donors, and treating the donor blood plasma to obtain immunoglobulin in a purified state having an antibody titer to the SdrX protein which is higher than that found in pooled intravenous immunoglobulin obtained from unselected human donors.

33. The purified human donor plasma immunoglobulin composition of claim 31 wherein said composition is obtained by a method comprising administering an S. capitis SdrX protein to a human host donor in an amount sufficient to induce an antibody titer to the S. capitis SdrX protein that is higher than that found in pooled intravenous immunoglobulin obtained from unselected donors, recovering blood or plasma samples from the induced donors, and treating the donor blood or plasma to obtain immunoglobulin in a purified state having antibody titer to an S. capitis SdrX protein in an amount which is higher than that found in pooled intravenous immunoglobulin obtained from unselected human donors.
